(19) United States
(12) Patent Application Publication
(10) Pub. No.: US 2003/0175360 A1
(43) Pub. Date: Sep. 18, 2003

(54) Symptomatic relief of gastrointestinal disorders

(76) Inventor: Renzo Luzzatti, Weston, FL (US)

Correspondence Address: BANNER & WITCOFF, 1001 G STREET N W, SUITE 1100, WASHINGTON, DC, 20001, US

(21) Appl. No.: 10/079,569

(22) Filed: Feb. 22, 2002

(51) Int. Cl. A61K 31/46; A61K 31/29; A61K 33/08; A61K 33/06; A61K 33/24

(52) U.S. Cl. 424/653; 424/682; 424/691; 514/304; 514/503; 514/537

(57) ABSTRACT

A formulation for treating a gastrointestinal disorder is provided. The formulation provides symptomatic relief of symptoms associated with gastrointestinal disorders. Additionally, a method for treating a gastrointestinal disorder comprising administering a therapeutically effective amount of the formulation is provided.

### FIELD OF THE INVENTION

The subject invention is directed to a formulation and a method for using the same for treating a gastrointestinal disorder. More particularly, the subject invention is directed to a formulation for relief of symptoms associated with a gastrointestinal disorder.

### BACKGROUND OF THE INVENTION

The gastrointestinal digestive system functions to breakdown and digest food to release nutrients. Along its path from the stomach, to the small intestine, to the large intestine, and to its ultimate expulsion, food is broken down and digested in a series of chemical and enzymatic reactions to release needed nutrients. Food flows in one direction, through a series of specialized compartments, which extends from the mouth to the rectum.

Reverse flow of acids and other contents in the gastrointestinal tract (GI) is one basis for numerous GI diseases and disorders. When flow is reversed, the contents of a downstream specialized compartment are spilled into an upstream compartment. The upstream compartments are usually ill-equipped to handle the downstream contents. Pain and/or discomfort may result as the tissue and lining of an upstream specialized compartment is damaged and/or destroyed by downstream contents.

Gastroesophageal reflux disease (GERD) is a disease where the contents of the stomach flow back upstream into the esophagus. The lining of the esophagus is delicate and is not equipped to handle the acidic (i.e., low pH) contents from the stomach. The lining of the esophagus is burned by the stomach acid, causing pain and/or discomfort. A hallmark feature of GERD is a burning sensation in the throat. The pain and/or discomfort is often termed acid reflux or heartburn.

Typically, damaged esophageal lining will repair itself, but the associated pain and or discomfort will persist until the repair is complete. Many treatments have been proposed for preventing or reducing the backflow of acidic contents of the stomach into the esophagus for minimizing the associated discomfort and/or damage. Exemplary methods are disclosed in U.S. Pat. Nos. 6.251.063; 6.238.335; 6,197,331; 6,156,771; 6,098,629; 5,955,097; 5,877.192; 5.730.958; 5.719.197; and 5,254,591. However, there is a need for a formulation and a method using the same for providing improved symptomatic relief of acid reflux, heartburn (and/or other undesirable symptoms) associated with many gastrointestinal diseases and/or disorders, especially GERD, gastrointestinal irritation, gastrointestinal inflammation, and gastrointestinal infection.

### BRIEF SUMMARY OF THE INVENTION

It is thus an object of this invention to provide a formulation for treating a gastrointestinal disorder. It is a further object of this invention to provide a formulation for the symptomatic relief of pain and/or discomfort associated with GERD.

It is another object of this invention to provide a method for treating a gastrointestinal disorder, and the pain or discomfort associated therewith, in a patient (e.g., a human or a veterinary animal) in need thereof.

These and other objects of the invention are provided by one or more embodiments described below. In one embodiment, a formulation for treating a gastrointestinal disorder is provided. The formulation comprises:

(a) a locally acting anesthetic, and

(b) an antacid.

In still another embodiment of the invention, a formulation for treating a gastrointestinal disorder is provided. The formulation comprises:

(a) at least two locally acting anesthetics.

According to another embodiment of the invention, a method for treating a gastrointestinal disorder in a patient in need thereof is provided. The method comprises the step of:

(a) administering to the patient a therapeutically effective amount of the above-noted formulations.

According to yet another embodiment of the invention, a method for treating a gastrointestinal disorder in a patient in need thereof is provided. The method comprises the step of:

(a) administering to the patient a therapeutically effective amount of a formulation comprising at least one locally acting anesthetic.

### DETAILED DESCRIPTION OF THE INVENTION

The term “locally acting anesthetic” means an anesthetic which acts at the site of application and/or the area immediately surrounding the site of application that provides anesthetic activity when applied to a surface located on or within a body. Examples of such surfaces include, but are not limited to those of the skin, tongue, pharynx, esophagus, stomach, small intestine, large intestine, and other gastrointestinal linings.

The term “alkaline buffering agent” means a compound which contains at least one hydroxyl group for interacting with hydrogen ions and increasing or stabilizing the pH.

The term “H2 blocker” means the pharmaceutical agent that blocks the histamine H2 receptor thereby reducing or eliminating the production of hydrochloric acid in the stomach.

The term “proton pump inhibitor” means the pharmaceutical agent that blocks the pumping of hydrogen ions from the parietal cells into the secretory canaliculi. thereby reducing or eliminating the production of hydrochloric acid in the stomach.

The term “antispasm/muscle relaxing agent” means a pharmaceutical agent that reduces the activity or relieves spasms of the unstriated muscle in the wall of the GI tract, or other muscles.

The term “muscle tone agent” means a prokinetic pharmaceutical agent that influences motility and/or muscle tone in the gastrointestinal tract (such as Cisapride) often via dopaminergic and/or 5HT3/serotonergic mechanisms.

The term “antifoaming agent” means an ingredient that reduces the interfacial tension between air and the liquid environment, thereby reducing or eliminating the bubbles that create the foam.

The term “lining” means the endothelial layer on the interior surface of the gastrointestinal tract. The “lining” may extend from the interior surface to a depth of, for example, about 0-2 mm.

The term “gastrointestinal tract” means the digestive system from the mouth to the rectum and anus. The digestive tract comprises the mouth, pharynx, upper and lower esophagus, including upper esophagus, lower esophagus, upper esophageal sphincter, lower esophageal sphincter, stomach, small intestine including ileum, duodenum, jejunum, and large intestine including ascending colon, transverse colon, descending colon, sigmoid colon, rectum and anus.

The term “symptomatic relief” means an agent that reduces or eliminates the perceived symptoms of a disease or other abnormal state.

The term “symptoms associated with” means those symptoms felt during an episode of a particular diseased state, for example; coughing, sneezing, running nose and fevers are associated with the flu, and pain is a symptom associated with the diseases commonly referred to as heartburn, or GERD or duodenal ulcers.

The term “surgical implant” means a device which is placed into the body, through surgery.

The term “slow release” means that the active pharmaceutical ingredient is released from the dosage form at a release rate that is slower than from an “immediate releasing” dosage form. The rate of release of the active pharmaceutical ingredient is controlled by the dosage form.

According to another embodiment of the invention, a formulation for treating a gastrointestinal disorder is provided. Such formulation comprises:

(a) a locally acting anesthetic, and

(b) an antacid.

Gastrointestinal disorders include, but are not limited to, reflux, ulcer, gastritis, dyspepsia, nausea, abrasion to gastrointestinal tract, heart burn, hiatal hernia, gastrointestinal abscess, inflammatory bowel disease, colitis, Crohn's disease, ileitis, ileocolitis, ulcerative proctitis, irritable bowel syndrome, gastroenteritis, diverticulitis, diverticulosis, and combinations thereof. More common gastrointestinal disorders include, but are not limited to, reflux, ulcer, gastritis, dyspepsia, and combinations thereof

Reflux usually includes, but is not limited to, gastrointestinal reflux disease (GERD), reflux esophagitis, reflux laryngitis, acid reflux, and combinations thereof.

Typically, an ulcer includes, but is not limited to, esophageal ulcer, gastric peptic ulcer, duodenal peptic ulcer, and combinations thereof.

An abrasion typically includes, but is not limited to, scrapes, punctures. surgical injury, etc., and combinations thereof.

Locally acting anesthetics suitable for use with the present invention include. but are not limited to, cocaine, cocaine hydrochloride, lignocaine, lignocaine hydrochloride bupivicaine, bupivicaine hydrochloride, oxethazaine, oxethazaine hydrochloride. dibucaine, dibucaine hydrochloride, lidocaine, lidocaine hydrochloride, benzocaine, dyclonine, dyclonine hydrochloride, p-buthylaminobenzoic acid 2-(diethylamino) ethyl ester, p-buthylaminobenzoic acid 2-(diethylamino) ethyl ester hydrochloride, procaine, procaine hydrochloride, tetracaine, tetracaine hydrochloride. chloroprocaine, chloroprocaine hydrochloride, oxyprocaine, oxyprocaine hydrochloride, mepivacaine, mepivacaine hydrochloride, piperocaine, piperocaine hydrochloride, pramoxine, pramoxine hydrochloride, chlorobutanol, benzyl alcohol, butacaine, and combinations thereof. Preferred locally acting anesthetics include, but are not limited to, lidocaine hydrochloride, benzyl alcohol, chlorobutanol. dibucaine, dyclonine, pramoxine, dibucaine hydrochloride, dyclonine hydrochloride, pramoxine hydrochloride, benzocaine, and combinations thereof. More preferred locally acting anesthetics include, but are not limited to, benzocaine, dibucaine, dyclonine, pramoxine, dibucaine hydrochloride, dyclonine hydrochloride, pramoxine hydrochloride, and combinations thereof. Even more preferred locally acting anesthetics include, but are not limited to, dibucaine hydrochloride, dyclonine, dyclonine hydrochloride, pramoxine hydrochloride, benzocaine, and combinations thereof. Yet even more preferred locally acting anesthetics include, but are not limited to, benzocaine, dyclonine, dyclonine hydrochloride, and combinations thereof.

The above-noted locally acting anesthetics are usually provided in an amount from about 0.01% to about 50% by weight based on a total weight of the formulation. Preferred amounts are from about 0.1% to about 25% by weight of the locally acting anesthetic based on a total weight of the formulation. More preferred amounts are from about 0.25% to about 10% by weight of the locally acting anesthetic based on a total weight of the formulation. Even more preferred amounts are from about 0.5% to about 5% by weight of the locally acting anesthetic based on a total weight of the formulation. Yet even more preferred amounts are from about 1% to about 2% by weight of the locally acting anesthetic based on a total weight of the formulation.

Antacids suitable for use with the present invention include, but are not limited to. aluminum carbonate, aluminum hydroxy carbonate, aluminum hydroxide, aluminum phosphate, aluminum citrate, dihydroxyaluminum sodium carbonate, aluminum magnesium glycinate, dihydroxyaluminum aminoacetic acid, dihydroxyaluminum aminoacetate, bismuth aluminate, bismuth carbonate, bismuth subcarbonate, bismuth subgallate, bismuth subnitrate, calcium carbonate, calcium hydroxide, calcium phosphate, calcium citrate, calcium citrate malate, activated sulfate, magnesium aluminate, hydrated magnesium aluminate, magnesium aluminosilicates, magnesium carbonate, magnesium glycinate, magnesium hydroxide, magnesium oxide, magnesium trisilicate, potassium carbonate, potassium phosphate, potassium citrate, sodium carbonate, sodium bicarbonate, sodium phosphate, sodium citrate, and combinations thereof. Preferred antacids include, but are not limited to, hydrated magnesium aluminate, magnesium hydroxide, aluminum phosphate, calcium phosphate, magnesium carbonate, magnesium oxide, magnesium trisilicate, aluminum hydroxide, dihydroxy aluminum amino acetate, sodium bicarbonate, calcium carbonate, and combinations thereof. More preferred antacids include, but are not limited to, calcium phosphate, magnesium carbonate, magnesium oxide, magnesium trisilicate, aluminum hydroxide, dihydroxy aluminum amino acetate, sodium bicarbonate, calcium carbonate, and combinations thereof. Even more preferred antacids include, but are not limited to, aluminum hydroxide, dihydroxy aluminum amino acetate, sodium bicarbonate, calcium carbonate, and combinations thereof. Yet even more preferred antacids include, but are not limited to, calcium carbonate and magnesium hydroxide.

Typically, the above noted antacid(s) is/are provided in an amount from about 1 mEq to about 60 mEq. Preferred amounts are from about 2 mEq to about 50 mEq. More preferred amounts are from about 5 mEq to about 40 mEq. Even more preferred amounts are from about 10 mEq to about 30 mEq. Yet even more preferred amounts are from about 15 mEq to about 25 mEq.

According to another embodiment of the invention, the formulation is provided in a dosage form compatible with medical applications. Examples of such dosage forms include, but are not limited to, elixirs, liquids, solutions, suspensions. emulsions, tablets, compressed tablets, film coated tablets, chewable tablets, quick dissolve tablets, effervescent tablets, multi-layer tablets, bi-layer tablets: sustained-release tablets, other sustained release dosage form, (such as sustained-release capsules. sustained release granules), capsules, soft gelatin capsules, hard gelatin capsules. caplets, lozenges, chewable lozenges, beads, powders, granules, cachets, douches, suppository, cream, topical formulation, inhalant, patch, implant, depot implant, ingestible formulation, injectable formulation, infusion, food, a bar (such as health bar or candy bar), cereal, chewing gum, animal feed, drink and combinations thereof. Preferred dosage forms include, but are not limited to, elixirs, suspensions, emulsions, compressed tablets, capsules, soft gelatin capsules, effervescent tablets, chewing gums, quick dissolve tablets, chewable tablets, lozenges, and combinations thereof. More preferred dosage forms include, but are not limited to, compressed tablets. capsules, soft gelatin capsules, effervescent tablets, chewing gums, quick dissolve tablets, chewable tablets, lozenges, and combinations thereof. Even more preferred dosage forms include, but are not limited to, chewing gums, quick dissolve tablets, chewable tablets, lozenges, and combinations thereof. Yet even more preferred dosage forms include, but are not limited to, lozenges, liquids, and chewable tablets.

According to another embodiment of the invention, the formulation optionally further comprises a taste enhancer. Typical taste enhancers suitable for use with the present invention include, but are not limited to, acesulfame-K, aspartame, benzaldehyde, citric acid, corn syrup, fructose, glucose, maltol, mannitol, menthol, monosodium glutamate, saccharin, saccharin sodium, sodium chloride, sorbitol, sucralose, sucrose, vanillin, and combinations thereof. Preferred taste enhancers include, but are not limited to, menthol, monosodium glutamate, vanillin, citric acid, sodium chloride, mannitol, aspartame, saccharin sodium, acesulfame-K, sucrose, and combinations thereof. More preferred taste enhancers include, but are not limited to, citric acid, sodium chloride, mannitol, aspartame, saccharin sodium, acesulfame-K, sucrose, and combinations thereof. Even more preferred taste enhancers include, but are not limited to, aspartame, saccharin sodium, acesulfame-K, sucrose, and combinations thereof Yet even more preferred taste enhancers include, but are not limited to. sucrose.

The above noted taste enhancer(s) is/are provided in an amount from about 0.05%, to about 60% by weight based on a total weight of the formulation. Preferred amounts are from about 0.1% to about 40% by weight of the taste enhancer(s) based on a total weight of the formulation. More preferred amounts are from about 0.5% to about 25% by weight of the taste enhancer(s) based on a total weight of the formulation. Even more preferred amounts are from about 1% to about 10% by weight of the taste enhancer(s) based on a total weight of the formulation. Yet even more preferred amounts are from about 2% to about 5% by weight of the taste enhancer(s) based on a total weight of the formulation.

According to another embodiment of the invention, the formulation optionally further comprises a therapeutically effective amount of at least one drug to block stomach acid production or counter the effects of acid production or provide symptomatic relief of gastrointestinal disorders, e.g., minimize the amount of reflux of acidic stomach contents into the esophagus. Examples of such drugs include, but are not limited to, an H2 blocker, a proton pump inhibitor, an antispasm/muscle relaxant, a prokinetic and gastrokinetic agent, an antifoaming agent, anticholinergic agents and combinations thereof.

H2 blockers include, but are not limited to, famotidine, cimetidine, ranitidine, nizatidine, and combinations thereof. Preferred H2 blockers include, but are not limited to, cimetidine, famotidine, ranitidine, nizatidine and combinations thereof. More preferred H2 blockers include, but are not limited to, cimetidine, ranitidine, nizatidine and a combination thereof. Even more preferred H2 blockers include, but are not limited to, ranitidine and nizatidine.

Proton pump inhibitors include, but are not limited to, omeprazole, lanoprazole, pantoprozole, esomeprazole, rabeprazole, and combinations thereof. Preferred proton pump inhibitors include, but are not limited to, omeprazole, lanoprazole. pantoprozole, esomeprazole, rabeprazole and combinations thereof. More preferred proton pump inhibitors include, but are not limited to, omeprazole and rabeprazole.

Antispasm/muscle relaxing agents include, but are not limited to, baclofen and 4-amino-3-(4-chloropheyl)-butanoic acid.

Typical gastrokinetic and prokinetic agents include, but are not limited to, metaclopramide.

Antifoaming agents include, but are not limited to, sucrafate and carafate.

Typical anticholinergic agents include, but are not limited to, clidinium.

The above noted drug(s) is/are usually provided in an amount from about 5 mg to about 100 mg. Preferred amounts are from about 10 mg to about 80 mg. More preferred amounts are from about 20 mg to about 40 mg.

Typical other pharmaceutically acceptable excipients known in the art including but not limited to suitable amounts of preservatives, emulsifying agents, suspending agents, diluents, natural or artificial sweeteners, taste-masking agents, coloiing agents, and flavoring agents, to provide a palatable and pleasant looking final product that are capable of being commingled with each other together with at least one safe and effective active agent in a manner that does not have an interaction which would substantially reduce the safety or pharmaceutical efficacy of the compositions under ordinary use situations.

According to another embodiment of the invention, the formulation optionally further comprises a pharmaceutically acceptable bioadhesive or polymer. The pharmaceutically acceptable bloadhesive or polymer is one that is sufficient to bind to the lining of a gastrointestinal tract, including, but not limited to, the interior lining of the mouth, pharynx, upper and lower gastrointestinal tract including upper esophagus, lower esophagus, upper esophageal sphincter, lower esophageal sphincter, stomach, small intestine including, ileum, duodenum, jejunum, large intestine including ascending colon, transverse colon, descending colon, sigmoid colon, rectum, and anus.

Some of the above-noted bioadhesive or polymer change their viscosity with a change in pH. Typically, the viscosity may either increase or decrease with an associated increase or decrease in pH. This property can be used to target the bioadhesive or polymer to GI lining in a particular portion of the gastrointestinal tract. For example, the bioadhesive or polymer may be targeted to the lower esophageal sphincter by utilizing an adhesive that increases viscosity with a decrease in pH. Thus. for example, as the bioadhesive or polymer travels from the upper esophageal sphincter to the lower esophageal sphincter, a decrease in pH will cause an increase in the adhesive's viscosity and result in its retention at or around the lower esophageal sphincter.

Typical pharmaceutically acceptable bioadhesives or polymers suitable for use with the present invention include, but are not limited to, cellulostic derivatives, polysaccharides, polypeptides, synthetic polymers, vinyl and acrylic derivatives, and other synthetic polymers. Cellulostic derivatives suitable for use with the present invention usually include, but are not limited to methyl cellulose, sodium carboxymethyl cellulose, hydroxyethyl cellulose, hydroxypropyl cellulose, and hydroxypropyl methylcellulose. Polysaccharides suitable for use with the present invention typically include, but are not limited to, acacia, agar, carageenan, pectin. sodium alginate, tragacanth, and xanthan gum. Typical polypeptides suitable for use with the present invention include, but are not limited to, casein, gelatin, and protamine sulfate. Vinyl and acrylic derivatives suitable for use with the present invention typically include, but are not limited to, polyvinyl alcohol, polyvinylpyrrolidone, carbomer, and polymethacrylates. Other synthetic polymers suitable for use with the present invention include, but are not limited to, polyethylene oxide and polyethylene glycol. Preferred pharmaceutically acceptable bioadhesives or polymers include, but are not limited to, acacia, tragacanth, gelatin, polyvinyl alcohol, sodium alginate, pectin, hydroxypropyl methylcellulose, hydroxypropyl cellulose, methylcellulose, carbomer, sodium carboxymethyl cellulose, xanthan gum. polyethylene oxide, polyethylene glycol and combinations thereof. More preferred pharmaceutically acceptable bioadhesives or polymers include, but are not limited to. polyvinyl alcohol, sodium alginate, pectin, hydroxypropyl methylcellulose. hydroxypropyl cellulose, methylcellulose, carbomer, sodium carboxymethyl cellulose. xanthan gum, polyethylene oxide, polyethylene glycol and combinations thereof. Even more preferred pharmaceutically acceptable bioadhesives or polymers include, but are not limited to, hydroxypropyl methylcellulose, hydroxypropyl cellulose. methylcellulose, carbomer, sodium carboxymethyl cellulose, xanthan gum, polyethylene oxide, polyethylene glycol and combinations thereof. Yet even more preferred pharmaceutically acceptable bioadhesives or polymers include, but are not limited to, carbomer, sodium carboxymethyl cellulose, xanthan gum, polyethylene oxide, polyethylene glycol and combinations thereof.

Typically, the above-noted bioadhesives and polymers are provided in an amount from about 0.1% to about 60% by weight based on a total weight of the formulation. Preferred amounts are from about 1% to about 50% by weight of the bioadhesive(s) and polymer(s) based on a total weight of the formulation. More preferred amounts are from about 3% to about 40% by weight of the bioadhesive(s) and polymer(s) based on a total weight of the formulation. Even more preferred amounts are from about 5% to about 30% by weight of the bioadhesive(s) and polymer(s) based on a total weight of the formulation. Yet even more preferred amounts are from about 7% to about 20% by weight of the bioadhesive(s) and polymer(s) based on a total weight of the formulation.

The formulations noted above can be used in a method to treat a gastrointestinal disorder. In addition, a formulation comprising one locally acting anesthetic can be used to treat a gastrointestinal disorder. Such a method of treatment comprises administering a therapeutically effective amount of the above noted formulations, including a formulation comprising one locally acting anesthetic. The administration can be through a route well known in the art of administering therapeutic agents. Examples of such routes include, but are not limited to, oral, injectable, rectal, and surgical. The surgical route may include a slow release or a fast release dosage implant. Gastrointestinal disorders amenable to treatment by the method include, but are not limited to, reflux, gastroesophageal reflux disease (GERD), reflux esophagitis. reflux laryngitis, antacid reflux, ulcer, esophageal ulcer, gastritis, dyspepsia, nausea. abrasion to gastrointestinal tract, scrapes, punctures, surgical injury, heart burn, hiatal hernia, gastrointestinal abscess, inflammatory bowel disease colitis. Crohn's disease. ileitis, ileocolitis, ulcerative proctitis, irritable bowel syndrome, gastroenteritis, diverticulitis, diverticulosis

According to another embodiment of the invention, a formulation for treating a gastrointestinal disorder is provided. Such formulation comprises:

(a) at least two locally acting anesthetics.

According to another embodiment of the invention, a method for treating a gastrointestinal disorder in a patient in need thereof is provided. Such method comprises the step of:

(a) administering to the patient a therapeutically effective amount of the above-noted formulations.

According to yet another embodiment of the invention, a method for treating a gastrointestinal disorder in a patient in need thereof is provided. Such a method comprises the step of:

(a) administering to the patient a therapeutically effective amount of a formulation comprising a locally acting anesthetic.

According to another embodiment of the invention, the administering step of the above-noted methods are by a route compatible with medical applications. Examples of such routes include, but are not limited to, oral, rectal, surgical, and combinations thereof.

According to another embodiment of the invention, a formulation for treating a gastrointestinal disorder is provided. Such a formulation comprises:

(a) at least two locally acting anesthetics, and

(b) an acid blocking agent.

The following examples are provided for illustrative purposes only. The percent values in the examples below are percent by weight values based or, a total weight of the dosage formulation as noted in the following tables. Table 1 is a cross-reference index to the example dosage formulations provided below.

TABLE 1

| | | | | | Antacid or |
|---|---|---|---|---|---|
| Table reference | | | Dosage Form | Anesthetic | Therapeutic drug |
| 1 | a | 1 | Lozenge | Anesthetic | |
| | | 2 | Lozenge | Anesthetic | Antacid |
| | | 3 | Lozenge | Anesthetic | Therapeutic drug |
| | b | 1 | Liquid | Anesthetic | |
| | | 2 | Liquid | Anesthetic | Antacid |
| | | 3 | Liquid | Anesthetic | Therapeutic drug |
| | c | 1 | Chewable tablet | Anesthetic | |
| | | 2 | Chewable tablet | Anesthetic | Antacid |
| | | 3 | Chewable tablet | Anesthetic | Therapeutic drug |
| 2 | a | 1 | Bioadhesive | Anesthetic | |
| | | 2 | Bioadhesive | Anesthetic | Antacid |
| | | 3 | Bioadhesive | Anesthetic | Therapeutic drug |

TABLE 1a1
LOZENGE WITH ANESTHETIC

| | | FORMULA | | FORMULA | | FORMULA | | FORMULA | |
|---|---|---|---|---|---|---|---|---|---|
| | | #1a1a | | #1a1b | | #1a1c | | #1a1d | |
| | | MG PER | % PER | MG PER | % PER | MG PER | % PER | MG PER | % PER |
| INGREDIENT | FUNCTION | LOZENGE | LOZENGE | LOZENGE | LOZENGE | LOZENGE | LOZENGE | LOZENGE | LOZENGE |
| Benzocaine | Anesthetic | 100.00 | 5.00 | | | | | | |
| Hydrochloride | | | | | | | | | |
| Menthol | Anesthetic | | | 14.00 | 0.70 | | | | |
| Dyclonine | Anesthetic | | | | | 10.00 | 0.50 | | |
| Hydrochloride | | | | | | | | | |
| Lidocaine | Anesthetic | | | | | | | 10.00 | 0.50 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Sucrose | Filler/ | 863.00 | 43.15 | 906.00 | 45.30 | 909.00 | 45.45 | 909.00 | 45.45 |
| | Sweetener | | | | | | | | |
| Mannitol | Filler | 863.00 | 43.15 | 907.00 | 45.35 | 910.00 | 45.50 | 910.00 | 45.50 |
| Sodium | Sweetener | 20.00 | 1.00 | 20.00 | 1.00 | 20.0 | 1.00 | 20.0 | 1.00 |
| Saccharin | | | | | | | | | |
| Polyvinyl | Binder | 100.00 | 5.00 | 100.00 | 5.00 | 100.0 | 5.00 | 100.0 | 5.00 |
| Silicon | Glidant | 10.00 | 0.50 | 10.00 | 0.50 | 10.0 | 0.50 | 10.0 | 0.50 |
| Dioxide | | | | | | | | | |
| Peppermint, | Flavor | 20.00 | 1.00 | 20.00 | 1.00 | | | 20.0 | 1.00 |
| spray dried | | | | | | | | | |
| Cherry, | Flavor | | | | | 20.0 | 1.00 | | |
| Spray dried | | | | | | | | | |
| FD&C Blue | Coloring | 6.00 | 0.30 | 8.00 | 0.40 | | | 3.00 | 0.15 |
| #1 Lake dye | agent | | | | | | | | |
| FD&C Yellow | Coloring | 3.00 | 0.15 | | | | | | |
| #6 Lake dye | agent | | | | | | | | |
| D&C Red | Coloring | | | | | 6.00 | 0.30 | 3.00 | 0.15 |
| #33 Lake dye | agent | | | | | | | | |
| Magnesium | Lubricant | 15.00 | 0.75 | 15.00 | 0.75 | 15.00 | 0.75 | 15.00 | 0.75 |
| stearate | | | | | | | | | |
| TOTAL | | 2000.0 | 100.0 | 2000.00 | 100.00 | 2000.00 | 100.00 | 2000.00 | 100.00 |

| | | FORMULA | | FORMULA | | FORMULA | |
|---|---|---|---|---|---|---|---|
| | | #1a1e | | #1a1f | | #1a1g | |
| | | MG PER | % PER | MG PER | % PER | MG PER | % PER |
| INGREDIENT | FUNCTION | LOZENGE | LOZENGE | LOZENGE | LOZENGE | LOZENGE | LOZENGE |
| Benzocaine | Anesthetic | | | | | 100 | 5.00 |
| Hydrochloride | | | | | | | |
| Menthol | Anesthetic | | | | | | |
| Dyclonine | Anesthetic | | | | | 5.00 | 0.25 |
| Hydrochloride | | | | | | | |
| Lidocaine | Anesthetic | | | | | | |
| Hydrochloride | | | | | | | |
| Procaine | Anesthetic | 10.00 | 0.50 | | | | |
| Hydrochloride | | | | | | | |
| Tetracaine | Anesthetic | | | 30.00 | 1.50 | 10.00 | 0.50 |
| Hydrochloride | | | | | | | |
| Sucrose | Filler/ | 909.00 | 45.45 | 899.00 | 44.95 | 854.00 | 42.70 |
| | Sweetener | | | | | | |
| Mannitol | Filler | 910.00 | 45.50 | 900.00 | 45.00 | 860.00 | 43.00 |
| Sodium | Sweetener | 20.0 | 1.00 | 20.0 | 1.00 | 20.0 | 1.00 |
| Saccharin | | | | | | | |
| Polyvinyl | Binder | 100.0 | 5.00 | 100.0 | 5.00 | 100.0 | 5.00 |
| Silicon | Glidant | 10.0 | 0.50 | 10.0 | 0.50 | 10.0 | 0.50 |
| Dioxide | | | | | | | |
| Peppermint, | Flavor | 20.0 | 1.00 | 20.0 | 1.00 | 20.0 | 1.00 |
| spray dried | | | | | | | |
| Cherry, | Flavor | | | | | | |
| Spray dried | | | | | | | |
| FD&C Blue | Coloring | | | | | | |
| #1 Lake dye | agent | | | | | | |
| FD&C Yellow | Coloring | 6.00 | 0.30 | 3.00 | 0.15 | 3.00 | 0.15 |
| #6 Lake dye | agent | | | | | | |
| D&C Red | Coloring | | | 3.00 | 0.15 | 3.00 | 0.15 |
| #33 Lake dye | agent | | | | | | |
| Magnesium | Lubricant | 15.00 | 0.75 | 15.00 | 0.75 | 15.00 | 0.75 |
| stearate | | | | | | | |
| TOTAL | | 2000.00 | 100.00 | 2000.00 | 100.00 | 2000.00 | 100.00 |

TABLE 1a2

| | | FORMULA | | FORMULA | | FORMULA | | FORMULA | |
|---|---|---|---|---|---|---|---|---|---|
| | | #1a2a | | #1a2b | | #1a2c | | #1a2d | |
| | | MG PER | % PER | MG PER | % PER | MG PER | % PER | MG PER | % PER |
| INGREDIENT | FUNCTION | LOZENGE | LOZENGE | LOZENGE | LOZENGE | LOZENGE | LOZENGE | LOZENGE | LOZENGE |
| Benzocaine | Anesthetic | 100.00 | 4.00 | | | | | | |
| Hydrochloride | | | | | | | | | |
| Menthol | Anesthetic | | | 14.00 | 0.56 | | | | |
| Dyclonine | Anesthetic | | | | | 10.00 | 0.50 | | |
| Hydrochloride | | | | | | | | | |
| Lidocaine | Anesthetic | | | | | | | 10.00 | 0.40 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Calcium Carbonate, | Antacid | 526.32 | 21.05 | | | | | | |
| 95% Active | | | | | | | | | |
| Sodium | Antacid | | | 840.00 | 33.60 | | | | |
| Bicarbonate | | | | | | | | | |
| Aluminum | Antacid | | | | | 780.00 | 31.20 | | |
| Hydroxide | | | | | | | | | |
| Dihydroxyaluminum | Antacid | | | | | | | 1350.00 | 54.00 |
| Aminoacetic Acid | | | | | | | | | |
| Magnesium | Antacid | | | | | | | | |
| Hydroxide | | | | | | | | | |
| Aluminum | Antacid | | | | | | | | |
| Hydroxide/ | | | | | | | | | |
| Magnesium | | | | | | | | | |
| Hydroxide | | | | | | | | | |
| Dihydroxyaluminum | Antacid | | | | | | | | |
| Sodium Carbonate | | | | | | | | | |
| Sucrose | Filler/ | 826.20 | 33.05 | 718.00 | 28.72 | 750.00 | 30.00 | 463.50 | 18.54 |
| | Sweetener | | | | | | | | |
| Mannitol | Filler | 829.70 | 33.19 | 714.20 | 28.57 | 746.25 | 29.85 | 463.25 | 18.53 |
| Sodium Saccharin | Sweetener | 25.00 | 1.00 | 25.00 | 1.00 | 25.00 | 1.00 | 25.00 | 1.00 |
| Polyvinyl | Binder | 125.00 | 5.00 | 125.00 | 5.00 | 125.00 | 5.00 | 125.00 | 5.00 |
| pyrrolidone | | | | | | | | | |
| Silicon Dioxide | Glidant | 12.50 | 0.50 | 12.50 | 0.50 | 12.50 | 0.50 | 12.50 | 0.50 |
| Peppermint, | Flavor | 25.00 | 1.00 | 25.00 | 1.00 | | | 25.00 | 1.00 |
| spray dried | | | | | | | | | |
| Cherry, | Flavor | | | | | 25.00 | 1.00 | | |
| Spray dried | | | | | | | | | |
| FD&C Blue | Coloring | 7.50 | 0.30 | 7.50 | 0.30 | | | 3.50 | 0.14 |
| #1 Lake dye | agent | | | | | | | | |
| FD&C Yellow | Coloring | 4.00 | 0.16 | | | | | | |
| #6 Lake dye | agent | | | | | | | | |
| D&C Red | Coloring | | | | | 7.50 | 0.30 | 3.50 | 0.14 |
| #33 Lake dye | agent | | | | | | | | |
| Magnesium stearate | Lubricant | 18.75 | 0.75 | 18.75 | 0.75 | 18.75 | 0.75 | 18.75 | 0.75 |
| TOTAL | | 2499.97 | 100.00 | 2499.95 | 100.00 | 2500.00 | 100.10 | 2500.00 | 100.00 |

| | | FORMULA | | FORMULA | | FORMULA | |
|---|---|---|---|---|---|---|---|
| | | #1a2e | | #1a2f | | #1a2g | |
| | | MG PER | % PER | MG PER | % PER | MG PER | % PER |
| INGREDIENT | FUNCTION | LOZENGE | LOZENGE | LOZENGE | LOZENGE | LOZENGE | LOZENGE |
| Benzocaine | Anesthetic | | | | | 100.00 | 4.00 |
| Hydrochloride | | | | | | | |
| Menthol | Anesthetic | | | | | | |
| Dyclonine | Anesthetic | | | | | 5.00 | 0.20 |
| Hydrochloride | | | | | | | |
| Lidocaine | Anesthetic | | | | | | |
| Hydrochloride | | | | | | | |
| Procaine | Anesthetic | 10.00 | 0.40 | | | | |
| Tetracaine | Anesthetic | | | 30.00 | 1.20 | 10.00 | 0.40 |
| Hydrochloride | | | | | | | |
| Calcium Carbonate, | Antacid | | | | | | |
| 95% Active | | | | | | | |
| Sodium | Antacid | | | | | | |
| Bicarbonate | | | | | | | |
| Aluminum | Antacid | | | | | | |
| Hydroxide | | | | | | | |
| Dihydroxyaluminum | Antacid | | | | | | |
| Aminoacetic Acid | | | | | | | |
| Magnesium | Antacid | 583.00 | 23.32 | | | | |
| Hydroxide | | | | | | | |
| Aluminum | Antacid | | | 306.00 | 12.24 | | |
| Hydroxide/ | | | | | | | |
| Magnesium | | | | | | | |
| Hydroxide | | | | | | | |
| Dihydroxyaluminum | Antacid | | | | | 445.30 | 17.81 |
| Sodium Carbonate | | | | | | | |
| Sucrose | Filler/ | 845.00 | 33.80 | 970.00 | 38.80 | 857.70 | 34.31 |
| | Sweetener | | | | | | |
| Mannitol | Filler | 849.75 | 33.99 | 981.75 | 39.27 | 869.75 | 34.79 |
| Sodium Saccharin | Sweetener | 25.00 | 1.00 | 25.00 | 1.00 | 25.00 | 1.00 |
| Polyvinyl | Binder | 125.00 | 5.00 | 125.00 | 5.00 | 125.00 | 5.00 |
| pyrrolidone | | | | | | | |
| Silicon Dioxide | Glidant | 12.50 | 0.50 | 12.50 | 0.50 | 12.50 | 0.50 |
| Peppermint, | Flavor | 25.00 | 1.00 | | | 12.50 | 0.50 |
| spray dried | | | | | | | |
| Cherry, | Flavor | | | 25.00 | 1.00 | 12.50 | 0.50 |
| Spray dried | | | | | | | |
| FD&C Blue | Coloring | | | 3.00 | 0.12 | 3.00 | 0.12 |
| #1 Lake dye | agent | | | | | | |
| FD&C Yellow | Coloring | 6.00 | 0.24 | | | 3.00 | 0.12 |
| #6 Lake dye | agent | | | | | | |
| D&C Red | Coloring | | | 3.00 | 0.12 | | |
| #33 Lake dye | agent | | | | | | |
| Magnesium stearate | Lubricant | 18.75 | 0.75 | 18.75 | 0.75 | 18.75 | 0.75 |
| TOTAL | | 2500.00 | 100.00 | 2500.00 | 100.00 | 2500.00 | 100.00 |

TABLE 1a3

| | | FORMULA | | FORMULA | | FORMULA | | FORMULA | |
|---|---|---|---|---|---|---|---|---|---|
| | | #1a3a | | #1a3b | | #1a3c | | #1a3d | |
| | | MG PER | % PER | MG PER | % PER | MG PER | % PER | MG PER | % PER |
| INGREDIENT | FUNCTION | LOZENGE | LOZENGE | LOZENGE | LOZENGE | LOZENGE | LOZENGE | LOZENGE | LOZENGE |
| Benzocaine | Anesthetic | 100.00 | 5.00 | | | | | | |
| Hydrochloride | | | | | | | | | |
| Menthol | Anesthetic | | | 14.00 | 0.70 | | | | |
| Dyclonine | Anesthetic | | | | | 10.00 | 0.50 | | |
| Hydrochloride | | | | | | | | | |
| Lidocaine | Anesthetic | | | | | | | 10.00 | 0.50 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Omeprazole | Proton Pump | 40.00 | 2.00 | | | | | | |
| | Inhib. | | | | | | | | |
| Omeprazole | Proton Pump | | | 20.00 | 1.00 | | | | |
| | Inhib. | | | | | | | | |
| Lansoprazole | Proton Pump | | | | | 30.00 | 1.50 | | |
| | Inhib. | | | | | | | | |
| Pantoprozole | Proton Pump | | | | | | | 40.00 | 2.00 |
| | Inhib. | | | | | | | | |
| Esomeprazole | Proton Pump | | | | | | | | |
| | Inhib. | | | | | | | | |
| Esomeprazole | Proton Pump | | | | | | | | |
| | Inhib. | | | | | | | | |
| Rabeprazole | Proton Pump | | | | | | | | |
| | Inhib. | | | | | | | | |
| Sucrose | Filler/ | 843.00 | 42.15 | 896.00 | 44.80 | 899.00 | 44.95 | 889.00 | 44.45 |
| | Sweetener | | | | | | | | |
| Mannitol | Filler | 843.00 | 42.15 | 897.00 | 44.85 | 890.00 | 44.50 | 890.00 | 44.50 |
| Sodium Saccharin | Sweetener | 20.00 | 1.00 | 20.00 | 1.00 | 20.00 | 1.00 | 20.00 | 1.00 |
| Polyvinyl | Binder | 100.00 | 5.00 | 100.00 | 5.00 | 100.00 | 5.00 | 100.00 | 5.00 |
| pyrrolidone | | | | | | | | | |
| Silicon Dioxide | Glidant | 10.00 | 0.50 | 10.00 | 0.50 | 10.00 | 0.50 | 10.00 | 0.50 |
| Peppermint, | Flavor | 20.00 | 1.00 | 20.00 | 1.00 | | | 20.00 | 1.00 |
| spray dried | | | | | | | | | |
| Cherry, | Flavor | | | | | 20.00 | 1.00 | | |
| Spray dried | | | | | | | | | |
| FD&C Blue | Coloring | 6.00 | 0.30 | 8.00 | 0.40 | | | 3.00 | 0.15 |
| #1 Lake dye | agent | | | | | | | | |
| FD&C Yellow | Coloring | 3.00 | 0.15 | | | | | | |
| #6 Lake dye | agent | | | | | | | | |
| D&C Red | Coloring | | | | | 6.00 | 0.30 | 3.00 | 0.15 |
| #33 Lake dye | agent | | | | | | | | |
| Magnesium stearate | Lubricant | 15.00 | 0.75 | 15.00 | 0.75 | 15.00 | 0.75 | 15.00 | 0.75 |
| TOTAL | | 2000.00 | 100.00 | 2000.00 | 100.00 | 2000.00 | 100.00 | 2000.00 | 100.00 |

| | | FORMULA | | FORMULA | | FORMULA | |
|---|---|---|---|---|---|---|---|
| | | #1a3e | | #1a3f | | #1a3g | |
| | | MG PER | % PER | MG PER | % PER | MG PER | % PER |
| INGREDIENT | FUNCTION | LOZENGE | LOZENGE | LOZENGE | LOZENGE | LOZENGE | LOZENGE |
| Benzocaine | Anesthetic | | | | | 100.00 | 5.00 |
| Hydrochloride | | | | | | | |
| Menthol | Anesthetic | | | | | | |
| Dyclonine | Anesthetic | | | | | 5.00 | 0.25 |
| Hydrochloride | | | | | | | |
| Lidocaine | Anesthetic | | | | | | |
| Hydrochloride | | | | | | | |
| Procaine | Anesthetic | 10.00 | 0.50 | | | | |
| Hydrochloride | | | | | | | |
| Tetracaine | Anesthetic | | | 30.00 | 1.50 | 10.00 | 0.50 |
| Hydrochloride | | | | | | | |
| Omeprazole | Proton Pump | | | | | | |
| | Inhib. | | | | | | |
| Omeprazole | Proton Pump | | | | | | |
| | Inhib. | | | | | | |
| Lansoprazole | Proton Pump | | | | | | |
| | Inhib. | | | | | | |
| Pantoprozole | Proton Pump | | | | | | |
| | Inhib. | | | | | | |
| Esomeprazole | Proton Pump | 40.00 | 2.00 | | | | |
| | Inhib. | | | | | | |
| Esomeprazole | Proton Pump | | | 20.00 | 1.00 | | |
| | Inhib. | | | | | | |
| Rabeprazole | Proton Pump | | | | | 20.00 | 1.00 |
| | Inhib. | | | | | | |
| Sucrose | Filler/ | 889.00 | 44.45 | 889.00 | 44.45 | 849.00 | 42.45 |
| | Sweetener | | | | | | |
| Mannitol | Filler | 890.00 | 44.50 | 890.00 | 44.50 | 845.00 | 42.25 |
| Sodium Saccharin | Sweetener | 20.00 | 1.00 | 20.00 | 1.00 | 20.00 | 1.00 |
| Polyvinyl | Binder | 100.00 | 5.00 | 100.00 | 5.00 | 100.00 | 5.00 |
| pyrrolidone | | | | | | | |
| Silicon Dioxide | Glidant | 10.00 | 0.50 | 10.00 | 0.50 | 10.00 | 0.50 |
| Peppermint, | Flavor | 20.00 | 1.00 | 20.00 | 1.00 | 20.00 | 1.00 |
| spray dried | | | | | | | |
| Cherry, | Flavor | | | | | | |
| Spray dried | | | | | | | |
| FD&C Blue | Coloring | | | | | | |
| #1 Lake dye | agent | | | | | | |
| FD&C Yellow | Coloring | 6.00 | 0.30 | 3.00 | 0.15 | 3.00 | 0.15 |
| #6 Lake dye | agent | | | | | | |
| D&C Red | Coloring | | | 3.00 | 0.15 | 3.00 | 0.15 |
| #33 Lake dye | agent | | | | | | |
| Magnesium stearate | Lubricant | 15.00 | 0.75 | 15.00 | 0.75 | 15.00 | 0.75 |
| TOTAL | | 2000.00 | 100.00 | 2000.00 | 100.00 | 2000.00 | 100.00 |

TABLE 1b1

| | | FORMULA | | FORMULA | | FORMULA | | FORMULA | |
|---|---|---|---|---|---|---|---|---|---|
| | | #1b1a | | #1b1b | | #1b1c | | #1b1d | |
| | | MG | % | MG PER | % PER | MG PER | % PER | MG PER | % PER |
| INGREDIENT | FUNCTION | PER DOSE | PER DOSE | DOSE | DOSE | DOSE | DOSE | DOSE | DOSE |
| Benzocaine | Anesthetic | 100.00 | 1.67 | | | | | | |
| Hydrochloride | | | | | | | | | |
| Menthol | Anesthetic | | | 14.00 | 0.23 | | | | |
| Dyclonine | Anesthetic | | | | | 10.00 | 0.17 | | |
| Hydrochloride | | | | | | | | | |
| Lidocaine | Anesthetic | | | | | | | 10.00 | 0.17 |
| Hydrochloride | | | | | | | | | |
| Procaine | Anesthetic | | | | | | | | |
| Hydrochloride | | | | | | | | | |
| Tetracaine Hydrochloride | Anesthetic | | | | | | | | |
| Sucrose | Sweetener | 1500.00 | 25.00 | 1500.00 | 25.00 | 1500.00 | 25.00 | 1500.00 | 25.00 |
| Xylitol | Sweetener | 1500.00 | 25.00 | 1500.00 | 25.00 | 1500.00 | 25.00 | 1500.00 | 25.00 |
| Sodium Carboxymethyl Cellulose | Thickener | 30.00 | 0.50 | 30.00 | 0.50 | 30.00 | 0.50 | 30.00 | 0.50 |
| Glycerin | Solubilizer | 600.00 | 10.00 | 600.00 | 10.00 | 600.00 | 10.00 | 600.00 | 10.00 |
| Peppermint Flavor | Flavor | 1.20 | 0.02 | | | | | 1.20 | 0.02 |
| Cherry Flavor | Flavor | | | 1.20 | 0.02 | | | | |
| Grape Flavor | Flavor | | | | | 1.20 | 0.02 | | |
| FD&C Blue | Coloring | 0.60 | 0.01 | | | 0.60 | 0.01 | | |
| #1 Dye | agent | | | | | | | | |
| FD&C Yellow | Coloring | 0.60 | 0.01 | | | | | 1.20 | 0.02 |
| #6 Dye | agent | | | | | | | | |
| D&C Red | Coloring | | | 1.20 | 0.02 | 0.60 | 0.01 | | |
| #33 Dye | agent | | | | | | | | |
| Sodium Benzoate | Preservative | 6.00 | 0.10 | 6.00 | 0.10 | 6.00 | 0.10 | 6.00 | 0.10 |
| Purified Water | Solvent | 2261.60 | 37.69 | 2347.60 | 39.13 | 2351.60 | 39.19 | 2351.60 | 39.19 |
| Totals | | 6000.00 | 100.00 | 6000.00 | 100.00 | 6000.00 | 100.00 | 6000.00 | 100.00 |

| | | FORMULA | | FORMULA | | FORMULA | |
|---|---|---|---|---|---|---|---|
| | | #1b1e | | #1b1f | | #1b1g | |
| | | MG PER | % PER | MG PER | % PER | MG PER | % PER |
| INGREDIENT | FUNCTION | DOSE | DOSE | DOSE | DOSE | DOSE | DOSE |
| Benzocaine | Anesthetic | | | | | 100.00 | 1.67 |
| Hydrochloride | | | | | | | |
| Menthol | Anesthetic | | | | | | |
| Dyclonine | Anesthetic | | | | | 5.00 | 0.08 |
| Hydrochloride | | | | | | | |
| Lidocaine | Anesthetic | | | | | | |
| Hydrochloride | | | | | | | |
| Procaine | Anesthetic | 10.00 | 0.17 | | | | |
| Hydrochloride | | | | | | | |
| Tetracaine | Anesthetic | | | 30.00 | 0.50 | 10.00 | 0.17 |
| Hydrochloride | | | | | | | |
| Sucrose | Sweetener | 1500.00 | 25.00 | 1500.00 | 25.00 | 1500.00 | 25.00 |
| Xylitol | Sweetener | 1500.00 | 25.00 | 1500.00 | 25.00 | 1500.00 | 25.00 |
| Sodium Carboxymethyl Cellulose | Thickener | 30.00 | 0.50 | 30.00 | 0.50 | 30.00 | 0.50 |
| Glycerin | Solubilizer | 600.00 | 10.00 | 600.00 | 10.00 | 600.00 | 10.00 |
| Peppermint Flavor | Flavor | | | | | | |
| Cherry Flavor | Flavor | | | 1.20 | 0.02 | 0.60 | 0.01 |
| Grape Flavor | Flavor | 1.20 | 0.02 | | | 0.60 | 0.01 |
| FD&C Blue | Coloring | 1.20 | 0.02 | | | 0.40 | 0.01 |
| #1 Dye | agent | | | | | | |
| FD&C Yellow | Coloring | | | 0.60 | 0.01 | 0.40 | 0.01 |
| #6 Dye | agent | | | | | | |
| D&C Red | Coloring | | | 0.60 | 0.01 | 0.40 | 0.01 |
| #33 Dye | agent | | | | | | |
| Sodium Benzoate | Preservative | 6.00 | 0.10 | 0.10 | 0.00 | | |
| Purified Water | Solvent | 2351.60 | 39.19 | 2337.50 | 38.96 | 2252.60 | 37.54 |
| Totals | | 6000.00 | 100.00 | 6000.00 | 100.00 | 6000.00 | 100.00 |

TABLE 1b2

| | | FORMULA | | FORMULA | | FORMULA | | FORMULA | |
|---|---|---|---|---|---|---|---|---|---|
| | | #1b2a | | #1b2b | | #1b2c | | #1b2d | |
| | | MG PER | % PER | MG PER | % PER | MG PER | % PER | MG PER | % PER |
| INGREDIENT | FUNCTION | DOSE | DOSE | DOSE | DOSE | DOSE | DOSE | DOSE | DOSE |
| Benzocaine | Anesthetic | 100.00 | 0.50 | | | | | | |
| Hydrochloride | | | | | | | | | |
| Menthol | Anesthetic | | | 14.00 | 0.07 | | | | |
| Dyclonine | Anesthetic | | | | | 10.00 | 0.05 | | |
| Hydrochloride | | | | | | | | | |
| Lidocaine | Anesthetic | | | | | | | 10.00 | 0.05 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Calcium Carbonate, 95% Active | Antacid | 526.32 | 2.63 | | | | | | |
| Sodium Bicarbonate | Antacid | | | 840.00 | 4.20 | | | | |
| Aluminum | Antacid | | | | | 780.00 | 3.90 | | |
| Hydroxide | | | | | | | | | |
| Dihydroxyaluminum Aminoacetic Acid | Antacid | | | | | | | 1350.00 | 6.75 |
| Magnesium | Antacid | | | | | | | | |
| Hydroxide | | | | | | | | | |
| Aluminum | Antacid | | | | | | | | |
| Hydroxide/ | | | | | | | | | |
| Magnesium | | | | | | | | | |
| Hydroxide | | | | | | | | | |
| Dihydroxyaluminum | Antacid | | | | | | | | |
| Sodium Carbonate | | | | | | | | | |
| Sucrose | Sweetener | 4500.00 | 22.50 | 4500.00 | 22.50 | 4500.00 | 22.50 | 4500.00 | 22.50 |
| Xylitol | Sweetener | 4500.00 | 22.50 | 4500.00 | 22.50 | 4500.00 | 22.50 | 4500.00 | 22.50 |
| Sodium | Thickener | 100.00 | 0.50 | 100.00 | 0.50 | 100.00 | 0.50 | 100.00 | 0.50 |
| Carboxymethyl | | | | | | | | | |
| Cellulose | | | | | | | | | |
| Glycerin | Solubilizer | 1000.00 | 5.00 | 1000.00 | 5.00 | 1000.00 | 5.00 | 1000.00 | 5.00 |
| Peppermint Flavor | Flavor | 4.00 | 0.02 | | | | | 4.00 | 0.02 |
| Cherry Flavor | Flavor | | | 4.00 | 0.02 | | | | |
| Grape Flavor | Flavor | | | | | 4.00 | 0.02 | | |
| FD&C Blue #1 Dye | Coloring | 2.00 | 0.01 | | | 2.00 | 0.01 | | |
| | agent | | | | | | | | |
| FD&C Yellow | Coloring | 2.00 | 0.01 | | | | | 4.00 | 0.02 |
| #6 Dye | agent | | | | | | | | |
| D&C Red | Coloring | | | 2.00 | 0.01 | 2.00 | 0.01 | | |
| #33 Dye | agent | | | | | | | | |
| Sodium Benzoate | Preservative | 20.00 | 0.10 | 20.00 | 0.10 | 20.00 | 0.10 | 20.00 | 0.10 |
| Purified Water | Solvent | 9245.68 | 46.23 | 9020.00 | 45.10 | 9082.00 | 45.41 | 8512.00 | 42.56 |
| TOTALS | | 20000.00 | 100.00 | 20000.00 | 100.00 | 20000.00 | 100.00 | 20000.00 | 100.00 |

| | | FORMULA | | FORMULA | | FORMULA | |
|---|---|---|---|---|---|---|---|
| | | #1a2e | | #1a2f | | #1a2g | |
| | | MG PER | % PER | MG PER | % PER | MG PER | % PER |
| INGREDIENT | FUNCTION | DOSE | DOSE | DOSE | DOSE | DOSE | DOSE |
| Benzocaine | Anesthetic | | | | | 100.00 | 0.50 |
| Hydrochloride | | | | | | | |
| Menthol | Anesthetic | | | | | | |
| Dyclonine | Anesthetic | | | | | 5.00 | 0.03 |
| Hydrochloride | | | | | | | |
| Lidocaine | Anesthetic | | | | | | |
| Hydrochloride | | | | | | | |
| Procaine | Anesthetic | 10.00 | 0.05 | | | | |
| Hydrochloride | | | | | | | |
| Tetracaine | Anesthetic | | | 30.00 | 0.15 | 10.00 | 0.05 |
| Hydrochloride | | | | | | | |
| Calcium Carbonate, | Antacid | | | | | | |
| 95% Active | | | | | | | |
| Sodium Bicarbonate | Antacid | | | | | | |
| Aluminum | Antacid | | | | | | |
| Hydroxide | | | | | | | |
| Dihydroxyaluminum Aminoacetic Acid | Antacid | | | | | | |
| Magnesium | Antacid | 583.00 | 2.92 | | | | |
| Hydroxide | | | | | | | |
| Aluminum | Antacid | | | 306.00 | 1.53 | | |
| Hydroxide/ | | | | | | | |
| Magnesium | | | | | | | |
| Hydroxide | | | | | | | |
| Dihydroxyaluminum | Antacid | | | | | 445.30 | 2.23 |
| Sodium Carbonate | | | | | | | |
| Sucrose | Sweetener | 4500.00 | 22.50 | 4500.00 | 22.50 | 4500.00 | 22.50 |
| Xylitol | Sweetener | 4500.00 | 22.50 | 4500.00 | 22.50 | 4500.00 | 22.50 |
| Sodium | Thickener | 100.00 | 0.50 | 100.00 | 0.50 | 100.00 | 0.50 |
| Carboxymethyl | | | | | | | |
| Cellulose | | | | | | | |
| Glycerin | Solubilizer | 1000.00 | 5.00 | 1000.00 | 5.00 | 1000.00 | 5.00 |
| Peppermint Flavor | Flavor | | | | | | |
| Cherry Flavor | Flavor | | | 4.00 | 0.02 | 2.00 | 0.01 |
| Grape Flavor | Flavor | 4.00 | 0.02 | | | 2.00 | 0.01 |
| FD&C Blue #1 Dye | Coloring | 4.00 | 0.02 | | | 1.50 | 0.01 |
| | agent | | | | | | |
| FD&C Yellow | Coloring | | | 2.00 | 0.01 | 1.50 | 0.01 |
| #6 Dye | agent | | | | | | |
| D&C Red | Coloring | | | 2.00 | 0.01 | 1.50 | 0.01 |
| #33 Dye | agent | | | | | | |
| Sodium Benzoate | Preservative | 20.00 | 0.10 | 20.00 | 0.10 | 20.00 | 0.10 |
| Purified Water | Solvent | 9279.00 | 46.40 | 9536.00 | 47.68 | 9311.20 | 46.56 |
| TOTALS | | 20000.00 | 100.00 | 20000.00 | 100.00 | 20000.00 | 100.00 |

TABLE 1b3

| | | FORMULA | | FORMULA | | FORMULA | | FORMULA | |
|---|---|---|---|---|---|---|---|---|---|
| | | #1b3a | | #1b3b | | #1b3c | | #1b3d | |
| | | MG | % PER | MG PER | % PER | MG PER | % PER | MG PER | % PER |
| INGREDIENT | FUNCTION | PER DOSE | DOSE | DOSE | DOSE | DOSE | DOSE | DOSE | DOSE |
| Benzocaine | Anesthetic | 100.00 | 1.67 | | | | | | |
| Hydrochloride | | | | | | | | | |
| Menthol | Anesthetic | | | 14.00 | 0.23 | | | | |
| Dyclonine | Anesthetic | | | | | 10.00 | 0.17 | | |
| Hydrochloride | | | | | | | | | |
| Lidocaine | Anesthetic | | | | | | | 10.00 | 0.17 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Omeprazole | Proton Pump | 40.00 | 0.67 | | | | | | |
| | Inhib | | | | | | | | |
| Omeprazole | Proton Pump | | | 20.00 | 0.33 | | | | |
| | Inhib | | | | | | | | |
| Lansoprazole | Proton Pump | | | | | 30.00 | 0.50 | | |
| | Inhib | | | | | | | | |
| Pantoprozole | Proton Pump | | | | | | | 40.00 | 0.67 |
| | Inhib | | | | | | | | |
| Esomeprazole | Proton Pump | | | | | | | | |
| | Inhib | | | | | | | | |
| Esomeprazole | Proton Pump | | | | | | | | |
| | Inhib | | | | | | | | |
| Rabeprazole | Proton Pump | | | | | | | | |
| | Inhib | | | | | | | | |
| Sucrose | Sweetener | 1500.00 | 25.00 | 1500.00 | 25.00 | 1500.00 | 25.00 | 1500.00 | 25.00 |
| Xylitol | Sweetener | 1500.00 | 25.00 | 1500.00 | 25.00 | 1500.00 | 25.00 | 1500.00 | 25.00 |
| Sodium Carboxymethyl Cellulose | Thickener | 30.00 | 0.50 | 30.00 | 0.50 | 30.00 | 0.50 | 30.00 | 0.50 |
| Glycerin | Solubilizer | 600.00 | 10.00 | 600.00 | 10.00 | 600.00 | 10.00 | 600.00 | 10.00 |
| Peppermint Flavor | Flavor | 1.20 | 0.02 | | | | | 1.20 | 0.02 |
| Cherry Flavor | Flavor | | | 1.20 | 0.02 | | | | |
| Grape Flavor | Flavor | | | | | 1.20 | 0.02 | | |
| FD&C Blue #1 Dye | Coloring | 0.60 | 0.01 | | | 0.60 | 0.01 | | |
| | agent | | | | | | | | |
| FD&C Yellow | Coloring | 0.60 | 0.01 | | | | | 1.20 | 0.02 |
| #6 Dye | agent | | | | | | | | |
| D&C Red | Coloring | | | 1.20 | 0.02 | 0.60 | 0.01 | | |
| #33 Dye | agent | | | | | | | | |
| Sodium Benzoate | Preservative | 6.00 | 0.10 | 6.00 | 0.10 | 6.00 | 0.10 | 6.00 | 0.10 |
| Purified Water | Solvent | 2221.60 | 37.03 | 2327.60 | 38.79 | 2321.60 | 38.69 | 2311.60 | 38.53 |
| TOTALS | | 6000.00 | 100.00 | 6000.00 | 100.00 | 6000.00 | 100.00 | 6000.00 | 100.00 |

| | | FORMULA | | FORMULA | | FORMULA | |
|---|---|---|---|---|---|---|---|
| | | #1b3e | | #1b3f | | #1b3g | |
| | | MG PER | % PER | MG PER | % PER | MG PER | % PER |
| INGREDIENT | FUNCTION | DOSE | DOSE | DOSE | DOSE | DOSE | DOSE |
| Benzocaine | Anesthetic | | | | | 100.00 | 1.67 |
| Hydrochloride | | | | | | | |
| Menthol | Anesthetic | | | | | | |
| Dyclonine | Anesthetic | | | | | 5.00 | 0.08 |
| Hydrochloride | | | | | | | |
| Lidocaine | Anesthetic | | | | | | |
| Hydrochloride | | | | | | | |
| Procaine | Anesthetic | 10.00 | 0.17 | | | | |
| Hydrochloride | | | | | | | |
| Tetracaine | Anesthetic | | | 30.00 | 0.50 | 10.00 | 0.17 |
| Hydrochloride | | | | | | | |
| Omeprazole | Proton Pump | | | | | | |
| | Inhib | | | | | | |
| Omeprazole | Proton Pump | | | | | | |
| | Inhib | | | | | | |
| Lansoprazole | Proton Pump | | | | | | |
| | Inhib | | | | | | |
| Pantoprozole | Proton Pump | | | | | | |
| | Inhib | | | | | | |
| Esomeprazole | Proton Pump | 40.00 | 0.67 | | | | |
| | Inhib | | | | | | |
| Esomeprazole | Proton Pump | | | 20.00 | 0.33 | | |
| | Inhib | | | | | | |
| Rabeprazole | Proton Pump | | | | | 20.00 | 0.33 |
| | Inhib | | | | | | |
| Sucrose | Sweetener | 1500.00 | 25.00 | 1500.00 | 25.00 | 1500.00 | 25.00 |
| Xylitol | Sweetener | 1500.00 | 25.00 | 1500.00 | 25.00 | 1500.00 | 25.00 |
| Sodium Carboxymethyl Cellulose | Thickener | 30.00 | 0.50 | 30.00 | 0.50 | 30.00 | 0.50 |
| Glycerin | Solubilizer | 600.00 | 10.00 | 600.00 | 10.00 | 600.00 | 10.00 |
| Peppermint Flavor | Flavor | | | | | | |
| Cherry Flavor | Flavor | | | 1.20 | 0.02 | 0.60 | 0.01 |
| Grape Flavor | Flavor | 1.20 | 0.02 | | | 0.60 | 0.01 |
| FD&C Blue #1 Dye | Coloring | 1.20 | 0.02 | | | 0.40 | 0.01 |
| | agent | | | | | | |
| FD&C Yellow | Coloring | | | 0.60 | 0.01 | 0.40 | 0.01 |
| #6 Dye | agent | | | | | | |
| D&C Red | Coloring | | | 0.60 | 0.01 | 0.40 | 0.01 |
| #33 Dye | agent | | | | | | |
| Sodium Benzoate | Preservative | 6.00 | 0.10 | 6.00 | 0.10 | 6.00 | 0.10 |
| Purified Water | Solvent | 2311.60 | 38.53 | 2311.60 | 38.53 | 2226.60 | 37.11 |
| TOTALS | | 6000.00 | 100.00 | 6000.00 | 100.00 | 6000.00 | 100.00 |

TABLE 1c1

| | | FORMULA | | FORMULA | | FORMULA | | FORMULA | |
|---|---|---|---|---|---|---|---|---|---|
| | | #1c1a | | #1c1b | | #1c1c | | #1c1d | |
| | | MG | % PER | MG PER | % PER | MG PER | % PER | MG PER | % PER |
| INGREDIENT | FUNCTION | PER TABLET | TABLET | TABLET | TABLET | TABLET | TABLET | TABLET | TABLET |
| Benzocaine | Anesthetic | 100.00 | 5.00 | | | | | | |
| Hydrochloride | | | | | | | | | |
| Menthol | Anesthetic | | | 14.00 | 0.70 | | | | |
| Dyclonine | Anesthetic | | | | | 10.00 | 0.50 | | |
| Hydrochloride | | | | | | | | | |
| Lidocaine | Anesthetic | | | | | | | 10.00 | 0.50 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Sucrose | Filler/Sweetener | 823.00 | 41.15 | 866.00 | 43.30 | 869.00 | 43.45 | 869.00 | 43.45 |
| Mannitol | Filler | 823.00 | 41.15 | 867.00 | 43.35 | 870.00 | 43.50 | 870.00 | 43.50 |
| Sodium Saccharin | Sweetener | 20.00 | 1.00 | 20.00 | 1.00 | 20.00 | 1.00 | 20.00 | 1.00 |
| Polyvinyl | Binder | 100.00 | 5.00 | 100.00 | 5.00 | 100.00 | 5.00 | 100.00 | 5.00 |
| pyrrolidone | | | | | | | | | |
| Croscarmelose | Disintegrant | 80.00 | 4.00 | 80.00 | 4.00 | 80.00 | 4.00 | 80.00 | 4.00 |
| Silicon Dioxide | Glidant | 10.00 | 0.50 | 10.00 | 0.50 | 10.00 | 0.50 | 10.00 | 0.50 |
| Peppermint, | Flavor | 20.00 | 1.00 | 20.00 | 1.00 | | | 20.00 | 1.00 |
| spray dried | | | | | | | | | |
| Cherry, | Flavor | | | | | 20.00 | 1.00 | | |
| Spray dried | | | | | | | | | |
| FD&C Blue | Coloring agent | 6.00 | 0.30 | 8.00 | 0.40 | | | 3.00 | 0.15 |
| #1 Lake dye | | | | | | | | | |
| FD&C Yellow #6 Lake dye | Coloring | 3.00 | 0.15 | | | | | | |
| | agent | | | | | | | | |
| D&C Red | Coloring | | | | | 6.00 | 0.30 | 3.00 | 0.15 |
| #33 Lake dye | agent | | | | | | | | |
| Magnesium stearate | Lubricant | 15.00 | 0.75 | 15.00 | 0.75 | 15.00 | 0.75 | 15.00 | 0.75 |
| TOTAL | | 2000.00 | 100.00 | 2000.00 | 100.00 | 2000.00 | 100.00 | 2000.00 | 100.00 |

| | | FORMULA | | FORMULA | | FORMULA | |
|---|---|---|---|---|---|---|---|
| | | #1c1e | | #1c1f | | #1c1g | |
| | | MG PER | % PER | MG PER | % PER | MG PER | % PER |
| INGREDIENT | FUNCTION | TABLET | TABLET | TABLET | TABLET | TABLET | TABLET |
| Benzocaine | Anesthetic | | | | | 100.00 | 5.00 |
| Hydrochloride | | | | | | | |
| Menthol | Anesthetic | | | | | | |
| Dyclonine | Anesthetic | | | | | 5.00 | 0.25 |
| Hydrochloride | | | | | | | |
| Lidocaine | Anesthetic | | | | | | |
| Hydrochloride | | | | | | | |
| Procaine | Anesthetic | | | | | | |
| Hydrochloride | | | | | | | |
| Tetracaine | Anesthetic | 10.00 | 0.50 | 30.00 | 1.50 | 10.00 | 0.50 |
| Hydrochloride | | | | | | | |
| Sucrose | Filler/Sweetener | 869.00 | 43.45 | 859.00 | 42.95 | 814.00 | 40.70 |
| Mannitol | Filler | 870.00 | 43.50 | 860.00 | 43.00 | 820.00 | 41.00 |
| Sodium Saccharin | Sweetener | 20.00 | 1.00 | 20.00 | 1.00 | 20.00 | 1.00 |
| Polyvinyl | Binder | 100.00 | 5.00 | 100.00 | 5.00 | 100.00 | 5.00 |
| pyrrolidone | | | | | | | |
| Croscarmelose | Disintegrant | 80.00 | 4.00 | 80.00 | 4.00 | 80.00 | 4.00 |
| Silicon Dioxide | Glidant | 10.00 | 0.50 | 10.00 | 0.50 | 10.00 | 0.50 |
| Peppermint, | Flavor | 20.00 | 1.00 | 20.00 | 1.00 | 20.00 | 1.00 |
| spray dried | | | | | | | |
| Cherry, | Flavor | | | | | | |
| Spray dried | | | | | | | |
| FD&C Blue | Coloring agent | | | | | | |
| #1 Lake dye | | | | | | | |
| FD&C Yellow #6 Lake dye | Coloring agent | 6.00 | 0.30 | 3.00 | 0.15 | 3.00 | 0.15 |
| D&C Red | Coloring agent | | | 3.00 | 0.15 | 3.00 | 0.15 |
| #33 Lake dye | | | | | | | |
| Magnesium stearate | Lubricant | 15.00 | 0.75 | 15.00 | 0.75 | 15.00 | 0.75 |
| TOTAL | | 2000.00 | 100.00 | 2000.00 | 100.00 | 2000.00 | 100.00 |

TABLE 1c2

| | | FORMULA | | FORMULA | | FORMULA | | FORMULA | |
|---|---|---|---|---|---|---|---|---|---|
| | | #1c2a | | #1c2b | | #1c2c | | #1c2d | |
| | | MG PER | % PER | MG PER | % PER | MG PER | % PER | MG PER | % PER |
| INGREDIENT | FUNCTION | TABLET | TABLET | TABLET | TABLET | TABLET | TABLET | TABLET | TABLET |
| Benzocaine | Anesthetic | 100.00 | 4.00 | | | | | | |
| Hydrochloride | | | | | | | | | |
| Menthol | Anesthetic | | | 14.00 | 0.56 | | | | |
| Dyclonine | Anesthetic | | | | | 10.00 | 0.50 | | |
| Hydrochloride | | | | | | | | | |
| Lidocaine | Anesthetic | | | | | | | 10.00 | 0.40 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Calcium Carbonate, | Antacid | 526.32 | 21.05 | | | | | | |
| 95% Active | | | | | | | | | |
| Sodium Bicarbonate | Antacid | | | 840.00 | 33.60 | | | | |
| Aluminum | Antacid | | | | | 780.00 | 31.20 | | |
| Hydroxide | | | | | | | | | |
| Dihydroxyaluminum Aminoacetic | Antacid | | | | | | | 1350.00 | 54.00 |
| Acid | | | | | | | | | |
| Magnesium | Antacid | | | | | | | | |
| Hydroxide | | | | | | | | | |
| Aluminum | Antacid | | | | | | | | |
| Hydroxide/ | | | | | | | | | |
| Magnesium | | | | | | | | | |
| Hydroxide | | | | | | | | | |
| Dihydroxyaluminum | Antacid | | | | | | | | |
| Sodium Carbonate | | | | | | | | | |
| Sucrose | Filler/Sweetener | 776.20 | 31.05 | 668.00 | 26.72 | 700.00 | 28.00 | 413.50 | 16.54 |
| Mannitol | Filler | 779.70 | 31.19 | 664.20 | 26.57 | 696.25 | 27.85 | 413.25 | 16.53 |
| Sodium Saccharin | Sweetener | 25.00 | 1.00 | 25.00 | 1.00 | 25.00 | 1.00 | 25.00 | 1.00 |
| Polyvinyl | Binder | 125.00 | 5.00 | 125.00 | 5.00 | 125.00 | 5.00 | 125.00 | 5.00 |
| pyrrolidone | | | | | | | | | |
| Croscarmelose | Disintegrant | 100.00 | 4.00 | 100.00 | 4.00 | 100.00 | 4.00 | 100.00 | 4.00 |
| Silicon Dioxide | Glidant | 12.50 | 0.50 | 12.50 | 0.50 | 12.50 | 0.50 | 12.50 | 0.50 |
| Peppermint, | Flavor | 25.00 | 1.00 | 25.00 | 1.00 | | | 25.00 | 1.00 |
| spray dried | | | | | | | | | |
| Cherry, | Flavor | | | | | 25.00 | 1.00 | | |
| Spray dried | | | | | | | | | |
| FD&C Blue | Coloring | 7.50 | 0.30 | 7.50 | 0.30 | | | 3.50 | 0.14 |
| #1 Lake dye | agent | | | | | | | | |
| FD&C Yellow | Coloring | 4.00 | 0.16 | | | | | | |
| #6 Lake dye | agent | | | | | | | | |
| D&C Red | Coloring | | | | | 7.50 | 0.30 | 3.50 | 0.14 |
| #33 Lake dye | agent | | | | | | | | |
| Magnesium stearate | Lubricant | 18.75 | 0.75 | 18.75 | 0.75 | 1875 | 0.75 | 18.75 | 0.75 |
| TOTAL | | 2499.97 | 100.00 | 2499.95 | 100.00 | 2500.00 | 100.10 | 2500.00 | 100.00 |

| | | FORMULA | | FORMULA | | FORMULA | |
|---|---|---|---|---|---|---|---|
| | | #1c2e | | #1c2f | | #1c2g | |
| | | MG PER | % PER | MG PER | % PER | MG PER | % PER |
| INGREDIENT | FUNCTION | TABLET | TABLET | TABLET | TABLET | TABLET | TABLET |
| Benzocaine | Anesthetic | | | | | 100.00 | 4.00 |
| Hydrochloride | | | | | | | |
| Menthol | Anesthetic | | | 14.00 | 0.56 | | |
| Dyclonine | Anesthetic | | | | | 5.00 | 0.20 |
| Hydrochloride | | | | | | | |
| Lidocaine | Anesthetic | | | | | | |
| Hydrochloride | | | | | | | |
| Procaine | Anesthetic | 10.00 | 0.40 | | | | |
| Hydrochloride | | | | | | | |
| Tetracaine | Anesthetic | | | 30.00 | 1.20 | 10.00 | 0.40 |
| Hydrochloride | | | | | | | |
| Calcium Carbonate, | Antacid | | | | | | |
| 95% Active | | | | | | | |
| Sodium Bicarbonate | Antacid | | | | | | |
| Aluminum | Antacid | | | | | | |
| Hydroxide | | | | | | | |
| Dihydroxyaluminum Aminoacetic | Antacid | | | | | | |
| Acid | | | | | | | |
| Magnesium | Antacid | 583.00 | 23.32 | | | | |
| Hydroxide | | | | | | | |
| Aluminum | Antacid | | | 306.00 | 12.24 | | |
| Hydroxide/ | | | | | | | |
| Magnesium | | | | | | | |
| Hydroxide | | | | | | | |
| Dihydroxyaluminum | Antacid | | | | | 445.30 | 17.81 |
| Sodium Carbonate | | | | | | | |
| Sucrose | Filler/Sweetener | 795.00 | 31.80 | 920.00 | 36.80 | 807.70 | 32.31 |
| Mannitol | Filler | 799.75 | 31.99 | 931.75 | 37.27 | 819.75 | 32.79 |
| Sodium Saccharin | Sweetener | 25.00 | 1.00 | 25.00 | 1.00 | 25.00 | 1.00 |
| Polyvinyl | Binder | 125.00 | 5.00 | 125.00 | 5.00 | 125.00 | 5.00 |
| pyrrolidone | | | | | | | |
| Croscarmelose | Disintegrant | 100.00 | 4.00 | 100.00 | 4.00 | 100.00 | 4.00 |
| Silicon Dioxide | Glidant | 12.50 | 0.50 | 12.50 | 0.50 | 12.50 | 0.50 |
| Peppermint, | Flavor | 25.00 | 1.00 | | | 12.50 | 0.50 |
| spray dried | | | | | | | |
| Cherry, | Flavor | | | 25.00 | 1.00 | 12.50 | 0.50 |
| Spray dried | | | | | | | |
| FD&C Blue | Coloring agent | | | 3.00 | 0.12 | 3.00 | 0.12 |
| #1 Lake dye | | | | | | | |
| FD&C Yellow | Coloring agent | 6.00 | 0.24 | | | 3.00 | 0.12 |
| #6 Lake dye | | | | | | | |
| D&C Red | Coloring agent | | | 3.00 | 0.12 | | |
| #33 Lake dye | | | | | | | |
| Magnesium stearate | Lubricant | 18.75 | 0.75 | 18.75 | 0.75 | 18.75 | 0.75 |
| TOTAL | | 2500.00 | 100.00 | 2500.00 | 100.00 | 2500.00 | 100.00 |

TABLE 1c3

| | | FORMULA | | FORMULA | | FORMULA | | FORMULA | |
|---|---|---|---|---|---|---|---|---|---|
| | | #1c3a | | #1c3b | | #1c3c | | #1c3d | |
| | | MG PER | % PER | MG PER | % PER | MG PER | % PER | MG PER | % PER |
| INGREDIENT | FUNCTION | TABLET | TABLET | TABLET | TABLET | TABLET | TABLET | TABLET | TABLET |
| Benzocaine | Anesthetic | 100.00 | 5.00 | | | | | | |
| Hydrochloride | | | | | | | | | |
| Menthol | Anesthetic | | | 14.00 | 0.70 | | | | |
| Dyclonine | Anesthetic | | | | | 10.00 | 0.50 | | |
| Hydrochloride | | | | | | | | | |
| Lidocaine | Anesthetic | | | | | | | 10.00 | 0.50 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Omeprazole | Proton Pump | 40.00 | 2.00 | | | | | | |
| | Inhib | | | | | | | | |
| Omeprazole | Proton Pump | | | 20.00 | 1.00 | | | | |
| | Inhib | | | | | | | | |
| Lansoprazole | Proton Pump | | | | | 30.00 | 1.50 | | |
| | Inhib | | | | | | | | |
| Panloprozole | Proton Pump | | | | | | | 40.00 | 2.00 |
| | Inhib | | | | | | | | |
| Esomeprazole | Proton Pump | | | | | | | | |
| | Inhib | | | | | | | | |
| Esomeprazole | Proton Pump | | | | | | | | |
| | Inhib | | | | | | | | |
| Rabeprazole | Proton Pump | | | | | | | | |
| | Inhib | | | | | | | | |
| Sucrose | Filler/Sweetener | 803.00 | 40.15 | 856.00 | 42.80 | 859.00 | 42.95 | 849.00 | 42.45 |
| Mannitol | Filler | 803.00 | 40.15 | 857.00 | 42.85 | 850.00 | 42.50 | 850.00 | 42.50 |
| Sodium Saccharin | Sweetener | 20.00 | 1.00 | 20.00 | 1.00 | 20.00 | 1.00 | 20.00 | 1.00 |
| Polyvinyl | Binder | 100.00 | 5.00 | 100.00 | 5.00 | 100.00 | 5.00 | 100.00 | 5.00 |
| pyrrolidone | | | | | | | | | |
| Croscarmelose | Disintegrant | 80.00 | 4.00 | 80.00 | 4.00 | 80.00 | 4.00 | 80.00 | 4.00 |
| Silicon Dioxide | Glidant | 10.00 | 0.50 | 10.00 | 0.50 | 10.00 | 0.50 | 10.00 | 0.50 |
| Peppermint, | Flavor | 20.00 | 1.00 | 20.00 | 1.00 | | | 20.00 | 1.00 |
| spray dried | | | | | | | | | |
| Cherry, | Flavor | | | | | 20.00 | 1.00 | | |
| Spray dried | | | | | | | | | |
| FD&C Blue | Coloring | 6.00 | 0.30 | 8.00 | 0.40 | | | 3.00 | 0.15 |
| #1 Lake dye | agent | | | | | | | | |
| FD&C Yellow #6 Lake dye | Coloring | 3.00 | 0.15 | | | | | | |
| | agent | | | | | | | | |
| D&C Red | Coloring | | | | | 6.00 | 0.30 | 3.00 | 0.15 |
| #33 Lake dye | agent | | | | | | | | |
| Magnesium stearate | Lubricant | 15.00 | 0.75 | 15.00 | 0.75 | 15.00 | 0.75 | 15.00 | 0.75 |
| TOTAL | | 2000.00 | 100.00 | 2000.00 | 100.00 | 2000.00 | 100.00 | 2000.00 | 100.00 |

| | | FORMULA | | FORMULA | | FORMULA | |
|---|---|---|---|---|---|---|---|
| | | #1c3e | | #1c3f | | #1c3g | |
| | | MG PER | % PER | MG PER | % PER | MG PER | % PER |
| INGREDIENT | FUNCTION | TABLET | TABLET | TABLET | TABLET | TABLET | TABLET |
| Benzocaine | Anesthetic | | | | | 100.00 | 5.00 |
| Hydrochloride | | | | | | | |
| Menthol | Anesthetic | | | | | | |
| Dyclonine | Anesthetic | | | | | 5.00 | 0.25 |
| Hydrochloride | | | | | | | |
| Lidocaine | Anesthetic | | | | | | |
| Hydrochloride | | | | | | | |
| Procaine | Anesthetic | 10.00 | 0.50 | | | | |
| Hydrochloride | | | | | | | |
| Tetracaine | Anesthetic | | | 30.00 | 1.50 | 10.00 | 0.50 |
| Hydrochloride | | | | | | | |
| Omeprazole | Proton Pump | | | | | | |
| | Inhib | | | | | | |
| Omeprazole | Proton Pump | | | | | | |
| | Inhib | | | | | | |
| Lansoprazole | Proton Pump | | | | | | |
| | Inhib | | | | | | |
| Panloprozole | Proton Pump | | | | | | |
| | Inhib | | | | | | |
| Esomeprazole | Proton Pump | 40.00 | 2.00 | | | | |
| | Inhib | | | | | | |
| Esomeprazole | Proton Pump | | | 20.00 | 1.00 | | |
| | Inhib | | | | | | |
| Rabeprazole | Proton Pump | | | | | 20.00 | 1.00 |
| | Inhib | | | | | | |
| Sucrose | Filler/Sweetener | 849.00 | 42.45 | 849.00 | 42.45 | 809.00 | 40.45 |
| Mannitol | Filler | 850.00 | 42.50 | 850.00 | 42.50 | 805.00 | 40.25 |
| Sodium Saccharin | Sweetener | 20.00 | 1.00 | 20.00 | 1.00 | 20.00 | 1.00 |
| Polyvinyl | Binder | 100.00 | 5.00 | 100.00 | 5.00 | 100.00 | 5.00 |
| pyrrolidone | | | | | | | |
| Croscarmelose | Disintegrant | 80.00 | 4.00 | 80.00 | 4.00 | 80.00 | 4.00 |
| Silicon Dioxide | Glidant | 10.00 | 0.50 | 10.00 | 0.50 | 10.00 | 0.50 |
| Peppermint, | Flavor | 20.00 | 1.00 | 20.00 | 1.00 | 20.00 | 1.00 |
| spray dried | | | | | | | |
| Cherry, | Flavor | | | | | | |
| Spray dried | | | | | | | |
| FD&C Blue | Coloring agent | | | | | | |
| #1 Lake dye | | | | | | | |
| FD&C Yellow #6 Lake dye | Coloring agent | 6.00 | 0.30 | 3.00 | 0.15 | 3.00 | 0.15 |
| D&C Red | Coloring agent | | | 3.00 | 0.15 | 3.00 | 0.15 |
| #33 Lake dye | | | | | | | |
| Magnesium stearate | Lubricant | 15.00 | 0.75 | 15.00 | 0.75 | 15.00 | 0.75 |
| TOTAL | | 2000.00 | 100.00 | 2000.00 | 100.00 | 2000.00 | 100.00 |

TABLE 2a1
BIOADHESIVE WITH ANESTHETIC

| | | FORMULA | | FORMULA | | FORMULA | | FORMULA | |
|---|---|---|---|---|---|---|---|---|---|
| | | #2a1a | | #2a1b | | #2a1c | | #2a1d | |
| | | MG PER | % PER | MG PER | % PER | MG PER | % PER | MG PER | % PER |
| INGREDIENT | FUNCTION | DOSE | DOSE | DOSE | DOSE | DOSE | DOSE | DOSE | DOSE |
| Benzocaine | Anesthetic | 100.0 | 2.0 | | | | | | |
| Hydrochloride | | | | | | | | | |
| Menthol | Anesthetic | | | 20.0 | 0.4 | | | | |
| Dyclonine | Anesthetic | | | | | 10.0 | 0.2 | | |
| Hydrochloride | | | | | | | | | |
| Lidocaine | Anesthetic | | | | | | | 100.0 | 2.0 |
| Hydrochloride | | | | | | | | | |
| Procaine | Anesthetic | | | | | | | | |
| Hydrochloride | | | | | | | | | |
| Tetracaine | Anesthetic | | | | | | | | |
| Hydrochloride | | | | | | | | | |
| Carbomer 974P | Gelling | 20.0 | 0.4 | | | | | | |
| | agent | | | | | | | | |
| Sodium Hydroxide | Alkalinizing agent | 2.0 | 0.0 | | | | | | |
| Sodium carboxymethyl cellulose | Thickener | | | 175.0 | 3.5 | | | | |
| Xanthan gum | Thickener | | | | | 100.0 | 2.0 | | |
| Sucralfale | Thickener | | | | | | | 500.0 | 10.0 |
| Carrageenan | Thickener | | | | | | | | |
| Sodium Alginate | Gelling | | | | | | | | |
| | agent | | | | | | | | |
| Polymethacrylate | Gelling | | | | | | | | |
| | agent | | | | | | | | |
| Peppermint Flavor | Flavor | 1.2 | 0.0 | | | | | | |
| Cherry Flavor | Flavor | | | 1.2 | 0.0 | | | | |
| Grape Flavor | Flavor | | | | | 1.2 | 0.0 | | |
| FD&C Blue | Coloring | 0.6 | 0.0 | | | 0.6 | 0.0 | | |
| #1 Dye | agent | | | | | | | | |
| FD&C Yellow | Coloring | 0.6 | 0.0 | | | | | 1.2 | 0.0 |
| #6 Dye | agent | | | | | | | | |
| D&C Red | Coloring | | | 1.2 | 0.0 | 0.6 | 0.0 | | |
| #33 Dye | agent | | | | | | | | |
| Sodium Benzoate | Preservative | 5.0 | 0.1 | 5.0 | 0.1 | 5.0 | 0.1 | 5.0 | 0.1 |
| Purified Water | Solvent | 4870.6 | 97.4 | 4797.6 | 96.0 | 4882.6 | 97.7 | 4392.6 | 87.9 |
| Totals | | 5000.0 | 100.0 | 5000.0 | 100.0 | 5000.0 | 100.0 | 5000.0 | 100.0 |

| | | FORMULA | | FORMULA | | FORMULA | |
|---|---|---|---|---|---|---|---|
| | | #2a1e | | #2a1f | | #2a1g | |
| | | MG PER | % PER | MG PER | % PER | MG PER | % PER |
| INGREDIENT | FUNCTION | DOSE | DOSE | DOSE | DOSE | DOSE | DOSE |
| Benzocaine | Anesthetic | | | | | 100.0 | 2.0 |
| Hydrochloride | | | | | | | |
| Menthol | Anesthetic | | | | | | |
| Dyclonine | Anesthetic | | | | | 10.2 | 0.2 |
| Hydrochloride | | | | | | | |
| Lidocaine | Anesthetic | | | | | | |
| Hydrochloride | | | | | | | |
| Procaine | Anesthetic | 50.0 | 1.0 | | | | |
| Hydrochloride | | | | | | | |
| Tetracaine | Anesthetic | | | 10.0 | 0.2 | | |
| Hydrochloride | | | | | | | |
| Carbomer 974P | Gelling agent | | | | | 20.0 | 0.4 |
| Sodium Hydroxide | Alkalinizing agent | | | | | 2.0 | 0.0 |
| Sodium | Thickener | | | | | | |
| carboxymethyl | | | | | | | |
| cellulose | | | | | | | |
| Xanthan gum | Thickener | | | | | | |
| Sucralfale | Thickener | | | | | | |
| Carrageenan | Thickener | 125.0 | 2.5 | | | | |
| Sodium Alginate | Gelling agent | | | 200.0 | 4.0 | | |
| Polymethacrylate | Gelling agent | | | | | 300.0 | 6.0 |
| Peppermint Flavor | Flavor | | | | | | |
| Cherry Flavor | Flavor | | | 1.2 | 0.0 | 0.6 | 0.0 |
| Grape Flavor | Flavor | 1.2 | 0.0 | | | 0.6 | 0.0 |
| FD&C Blue #1 Dye | Coloring agent | 1.2 | 0.0 | | | 0.4 | 0.0 |
| FD&C Yellow | Coloring | | | 0.6 | 0.0 | 0.4 | 0.0 |
| #6 Dye | agent | | | | | | |
| D&C Red | Coloring | | | 0.6 | 0.0 | 0.4 | 0.0 |
| #33 Dye | agent | | | | | | |
| Sodium Benzoate | Preservative | 5.0 | 0.1 | 5.0 | 0.1 | 5.0 | 0.1 |
| Purified Water | Solvent | 4817.6 | 96.4 | 4782.6 | 95.7 | 4560.6 | 91.2 |
| Totals | | 5000.0 | 100.0 | 5000.0 | 100.0 | 5000.0 | 100.0 |

TABLE 2a2
BIOADHESIVE WITH ANESTHETIC AND ANTACID

| | | FORMULA | | FORMULA | | FORMULA | | FORMULA | |
|---|---|---|---|---|---|---|---|---|---|
| | | #2a2a | | #2a2b | | #2a2c | | #2a2d | |
| | | MG PER | % PER | MG PER | % PER | MG PER | % PER | MG PER | % PER |
| INGREDIENT | FUNCTION | DOSE | DOSE | DOSE | DOSE | DOSE | DOSE | DOSE | DOSE |
| Benzocaine Hydrochloride | Anesthetic | 100.000 | 2.000 | | | | | | |
| Menthol | Anesthetic | | | 20.000 | 0.400 | | | | |
| Dyclonine Hydrochloride | Anesthetic | | | | | 10.000 | 0.200 | | |
| Lidocaine Hydrochloride | Anesthetic | | | | | | | 10.000 | 0.200 |
| Procaine Hydrochloride | Anesthetic | | | | | | | | |
| Tetracaine Hydrochloride | Anesthetic | | | | | | | | |
| Calcium Carbonate, 95% Active | Antacid | 526.316 | 10.526 | | | | | | |
| Sodium Bicarbonate | Antacid | | | 840.000 | 16.800 | | | | |
| Aluminum Hydroxide | Antacid | | | | | 780.000 | 15.600 | | |
| Dihydroxyaluminum Aminoacetic Acid | Antacid | | | | | | | 1350.000 | 27.000 |
| Magnesium Hydroxide | Antacid | | | | | | | | |
| Aluminum Hydroxide/Magnesium | Antacid | | | | | | | | |
| Hydroxide | | | | | | | | | |
| Dihydroxyaluminum Sodium | Antacid | | | | | | | | |
| Carbonate | | | | | | | | | |
| Carbomer 974P | Gelling | 20.000 | 0.400 | | | | | | |
| | agent | | | | | | | | |
| Sodium carboxymethyl cellulose | Thickener | | | 175.000 | 3.500 | | | | |
| Xanthan gum | Thickener | | | | | 100.000 | 2.000 | | |
| Sucralfate | Thickener | | | | | | | 500.000 | 10.000 |
| Carrageenan | Thickener | | | | | | | | |
| Sodium Alginate | Gelling | | | | | | | | |
| agent | | | | | | | | | |
| Polymethacrylate | Gelling | | | | | | | | |
| agent | | | | | | | | | |
| Peppermint Flavor | Flavor | 1.200 | 0.024 | | | | | 1.200 | 0.024 |
| Cherry Flavor | Flavor | | | 1.200 | 0.024 | | | | |
| Grape Flavor | Flavor | | | | | 1.200 | 0.024 | | |
| FD&C Blue #1 Dye | Coloring | 0.600 | 0.012 | | | 0.600 | 0.012 | | |
| agent | | | | | | | | | |
| FD&C Yellow #6 Dye | Coloring | 0.600 | 0.012 | | | | | 1.200 | 0.024 |
| agent | | | | | | | | | |
| D&C Red #33 Dye | Coloring | | | 1.200 | 0.024 | 0.600 | 0.012 | | |
| agent | | | | | | | | | |
| Sodium Benzoate | Preservative | 5.000 | 0.100 | 5.000 | 0.100 | 5.000 | 0.100 | 5.000 | 0.100 |
| Purified Water | Solvent | 4346.284 | 86.926 | 3957.600 | 79.152 | 4102.600 | 82.052 | 3132.600 | 62.652 |
| TOTALS | | 5000.000 | 100.000 | 5000.000 | 100.000 | 5000.000 | 100.000 | 5000.000 | 100.000 |

| | | FORMULA | | FORMULA | | FORMULA | |
|---|---|---|---|---|---|---|---|
| | | #2a2e | | #2a2f | | #2a2g | |
| | | MG PER | % PER | MG PER | % PER | MG PER | % PER |
| INGREDIENT | FUNCTION | DOSE | DOSE | DOSE | DOSE | DOSE | DOSE |
| Benzocaine Hydrochloride | Anesthetic | | | | | 100.000 | 2.000 |
| Menthol | Anesthetic | | | | | | |
| Dyclonine Hydrochloride | Anesthetic | | | | | 5.000 | 0.100 |
| Lidocaine Hydrochloride | Anesthetic | | | | | | |
| Procaine Hydrochloride | Anesthetic | 10.000 | 0.200 | | | | |
| Tetracaine Hydrochloride | Anesthetic | | | 30.000 | 0.600 | 10.000 | 0.200 |
| Calcium Carbonate, 95% Active | Antacid | | | | | | |
| Sodium Bicarbonate | Antacid | | | | | | |
| Aluminum Hydroxide | Antacid | | | | | | |
| Dihydroxyaluminum Aminoacetic Acid | Antacid | | | | | | |
| Magnesium Hydroxide | Antacid | 583.000 | 11.660 | | | | |
| Aluminum Hydroxide/Magnesium | Antacid | | | 306.000 | 6.120 | | |
| Hydroxide | | | | | | | |
| Dihydroxyaluminum Sodium | Antacid | | | | | 445.300 | 8.906 |
| Carbonate | | | | | | | |
| Carbomer 974P | Gelling | | | | | 20.000 | 0.400 |
| agent | | | | | | | |
| Sodium carboxymethyl cellulose | Thickener | | | | | | |
| Xanthan gum | Thickener | | | | | | |
| Sucralfate | Thickener | | | | | | |
| Carrageenan | Thickener | 125.000 | 2.500 | | | | |
| Sodium Alginate | Gelling | | | 200.000 | 4.000 | | |
| agent | | | | | | | |
| Polymethacrylate | Gelling | | | | | 300.0 | 6.0 |
| agent | | | | | | | |
| Peppermint Flavor | Flavor | | | | | | |
| Cherry Flavor | Flavor | | | 1.200 | 0.024 | 0.600 | 0012 |
| Grape Flavor | Flavor | 1.200 | 0.024 | | | 0.600 | 0.012 |
| FD&C Blue #1 Dye | Coloring | 1.200 | 0.024 | | | 0.400 | 0.008 |
| agent | | | | | | | |
| FD&C Yellow #6 Dye | Coloring | | | 0.600 | 0.012 | 0.400 | 0.008 |
| agent | | | | | | | |
| D&C Red #33 Dye | Coloring | | | 0.600 | 0.012 | 0.400 | 0.008 |
| agent | | | | | | | |
| Sodium Benzoate | Preservative | 5.000 | 0.100 | 5.000 | 0.100 | | |
| Purified Water | Solvent | 4274.600 | 85.492 | 4456.600 | 89.132 | 4117.300 | 82.346 |
| TOTALS | | 5000.000 | 100.000 | 5000.000 | 100.000 | 5000.000 | 100.000 |

TABLE 2a3
BIO ADHESIVE WITH ANESTHETIC AND PROTON PUMP INHIBITOR

| | | FORMULA | | FORMULA | | FORMULA | | FORMULA | |
|---|---|---|---|---|---|---|---|---|---|
| | | #2a3a | | #2a3b | | #2a3c | | #2a3d | |
| | | MG PER | % PER | MG PER | % PER | MG PER | % PER | MG PER | % PER |
| INGREDIENT | FUNCTION | DOSE | DOSE | DOSE | DOSE | DOSE | DOSE | DOSE | DOSE |
| Benzocaine Hydrochloride | Anesthetic | 100.00 | 2.00 | | | | | | |
| Menthol | Anesthetic | | | 14.00 | 0.280 | | | | |
| Dyclonine Hydrochloride | Anesthetic | | | | | 10.00 | 0.200 | | |
| Lidocaine Hydrochloride | Anesthetic | | | | | | | 10.00 | 0.200 |
| Procaine Hydrochloride | Anesthetic | | | | | | | | |
| Tetracaine Hydrochloride | Anesthetic | | | | | | | | |
| Omeprazole | Proton Pump Inhib | 40.00 | 0.80 | | | | | | |
| Omeprazole | Proton Pump Inhib | | | 20.00 | 0.400 | | | | |
| Lansoprazole | Proton Pump Inhib | | | | | 30.00 | 0.600 | | |
| Panloprozole | Proton Pump Inhib | | | | | | | 40.00 | 0.800 |
| Esomeprazole | Proton Pump Inhib | | | | | | | | |
| Esomeprazole | Proton Pump Inhib | | | | | | | | |
| Rabeprazole | Proton Pump Inhib | | | | | | | | |
| Carbomer 974P | Gelling agent | 20.0 | 0.4 | | | | | | |
| Sodium Hydroxide | Alkalinizing agent | 2.0 | 0.0 | | | | | | |
| Sodium carboxymethyl cellulose | Thickener | | | 175.0 | 3.500 | | | | |
| Xanthan gum | Thickener | | | | | 100.0 | 2.000 | | |
| Sucralfate | Thickener | | | | | | | 500.0 | 10.000 |
| Carrageenan | Thickener | | | | | | | | |
| Sodium Alginate | Gelling agent | | | | | | | | |
| Polymethacrylate | Gelling agent | | | | | | | | |
| Peppermint Flavor | Flavor | 1.200 | 0.02 | | | | | 1.200 | 0.024 |
| Cherry Flavor | Flavor | | | 1.200 | 0.024 | | | | |
| Grape Flavor | Flavor | | | | | 1.200 | 0.024 | | |
| FD&C Blue #1 Dye | Coloring agent | 0.600 | 0.01 | | | 0.600 | 0.012 | | |
| FD&C Yellow #6 Dye | Coloring agent | 0.600 | 0.01 | | | | | 1.200 | 0.024 |
| D&C Red #33 Dye | Coloring agent | | | 1.200 | 0.024 | 0.600 | 0.012 | | |
| Sodium Benzoate | Preservative | 5.00 | 0.10 | 5.00 | 0.100 | 5.00 | 0.100 | 5.00 | 0.100 |
| Purified Water | Solvent | 4830.60 | 96.61 | 4783.60 | 95.672 | 4852.60 | 97.052 | 4442.60 | 88.852 |
| TOTALS | | 5000.00 | 100.00 | 5000.00 | 100.00 | 5000.00 | 100.00 | 5000.00 | 100.00 |

| | | FORMULA | | FORMULA | | FORMULA | |
|---|---|---|---|---|---|---|---|
| | | #2a3e | | #2a3f | | #2a3g | |
| | | MG PER | % PER | MG PER | % PER | MG PER | % PER |
| INGREDIENT | FUNCTION | DOSE | DOSE | DOSE | DOSE | DOSE | DOSE |
| Benzocaine Hydrochloride | Anesthetic | | | | | 100.00 | 2.00 |
| Menthol | Anesthetic | | | | | | |
| Dyclonine Hydrochloride | Anesthetic | | | | | 5.00 | 0.10 |
| Lidocaine Hydrochloride | Anesthetic | | | | | | |
| Procaine Hydrochloride | Anesthetic | 10.00 | 0.200 | | | | |
| Tetracaine Hydrochloride | Anesthetic | | | 30.00 | 0.60 | 10.00 | 0.20 |
| Omeprazole | Proton Pump Inhib | | | | | | |
| Omeprazole | Proton Pump Inhib | | | | | 20.00 | 0.400 |
| Lansoprazole | Proton Pump Inhib | | | | | | |
| Panloprozole | Proton Pump Inhib | | | | | | |
| Esomeprazole | Proton Pump Inhib | 40.00 | 0.800 | | | | |
| Esomeprazole | Proton Pump Inhib | | | 20.00 | 0.40 | | |
| Rabeprazole | Proton Pump Inhib | | | | | 20.00 | 0.40 |
| Carbomer 974P | Gelling agent | | | | | 20.0 | 0.40 |
| Sodium Hydroxide | Alkalinizing agent | | | | | 2.0 | 0.04 |
| Sodium carboxymethyl cellulose | Thickener | | | | | | |
| Xanthan gum | Thickener | | | | | | |
| Sucralfate | Thickener | | | | | | |
| Carrageenan | Thickener | 125.0 | 2.500 | | | | |
| Sodium Alginate | Gelling agent | | | 200.0 | 4.00 | | |
| Polymethacrylate | Gelling agent | | | | | 300.0 | 6.00 |
| Peppermint Flavor | Flavor | | | | | | |
| Cherry Flavor | Flavor | | | 1.200 | 0.02 | 0.600 | 0.01 |
| Grape Flavor | Flavor | 1.200 | 0.024 | | | 0.600 | 0.01 |
| FD&C Blue #1 Dye | Coloring agent | 1.200 | 0.024 | | | 0.400 | 0.01 |
| FD&C Yellow #6 Dye | Coloring agent | | | 0.600 | 0.01 | 0.400 | 0.01 |
| D&C Red #33 Dye | Coloring agent | | | 0.600 | 0.01 | 0.400 | 0.01 |
| Sodium Benzoate | Preservative | 5.00 | 0.100 | 5.00 | 0.10 | 5.00 | 0.10 |
| Purified Water | Solvent | 4817.60 | 96.352 | 4742.60 | 94.85 | 4535.60 | 90.71 |
| TOTALS | | 5000.000 | 100.00 | 5000.00 | 100.00 | 5000.00 | 100.00 |

### REFERENCES

Silverman et al., U.S. Pat. No. 6,251,063 “Method for treating wall forming gastrointestinal tract.”

Silverman et al., U.S. Pat. No. 6,238,335 “Method for treating gastroesophageal reflux disease and apparatus for use therewith.”

Lerner et al., U.S. Pat. No. 6,197,331 “Pharmaceutical oral patch for controlled release of pharmaceutical agents in the oral cavity.”

Rubin et al., U.S. Pat. No. 6,156,771 “Method for alleviation of lower gastrointestinal disorders in a human patient.”

Johnson et al., U.S. Pat. No. 6.098,629 “Submucosal esophageal bulking device.”

Tapolsky et al., U.S. Pat. No. 5,955,097 “Pharmaceutical preparation applicable to mucosal surfaces and body tissues.”

Lindberg et al., U.S. Pat. No. 5,877,192 “Method for the treatment of gastric acid-related diseases and production of medication using (−) enantiomer of omeprazole.”

Sorosiek et al., U.S. Pat. No. 5,730,958 “Method of treatment of gastioesophageal reflux disease by enhancement of salivary esophageal protection due to mastication.”

Kanios et al, U.S. Pat. No. 5,719,197 “Compositions and methods for topical administration of pharmaceutically active agents.”

Martin et al., U.S. Pat. No. 5,254,591 “Pharmaceutical composition for treating gastroesophageal reflux.”

Remington's Pharmaceutical Sciences, 16^{th} ed., Mack Publishing Company, Easton P A. A. Osol, editor, (1980).

All references, articles, patents, patent applications, patent publications. textbooks and any other references cited in this application are incorporated herein by reference in their entirety.

I claim:

1. A formulation for treating a gastrointestinal disorder comprising:
a1) a locally acting anesthetic, and
b1) an antacid.

2. The formulation of claim 1 wherein said gastrointestinal disorder is selected from the group consisting of:
a2) reflux,
b2) ulcer,
c2) nausea,
d2) gastritis,
e2) dyspepsia,
P2) abrasion to gastrointestinal tract,
g2) heart burn,
h2) hiatal hernia,
i2) gastrointestinal abscess,
j2) inflammatory bowel disease.
k2) colitis,
l2) Crohn's disease,
m2) ileitis,
n2) ileocolitis,
o2) ulcerative proctitis,
p2) irritable bowel syndrome,
q2) gastroenteritis,
r2) diverticulitis,
s2) diverticulosis, and
t2) combinations thereof.

3. The formulation of claim 2
wherein said reflux (a2) is selected from the group consisting of:
a3) gastroesophageal reflux disease (GERD),
b3) reflux esophagitis.
c3) reflux laryngitis,
d3) acid reflux; and
wherein said ulcer (b2) is selected from the group consisting of:
e3) esophageal ulcer,
f3) gastric peptic ulcer, and
g3) duodenal peptic ulcer; and
wherein said abrasion (e2) to gastrointestinal tract is selected from the group consisting of:
h3) scrapes,
i3) puncture, and
j3) surgical; and
wherein said colitis (j2) comprises ulcerative colitis.

4. The formulation of claim 3 wherein said gastrointestinal disorder is gastroesophageal reflux disease (GERD).

5. The formulation of claim 3 wherein said gastrointestinal disorder is acid reflux (d3).

6. The formulation of claim 1 wherein said locally acting anesthetic (a1) is selected from the group consisting of:
a6) cocaine,
b6) lignocaine,
c6) bupivicaine,
d6) oxethazaine,
e6) dibucaine,
f6) lidocaine,
g6) benzocaine,
h6) dyclonine,
i6) p-buthylaminobenzoic acid 2-(diethylamino) ethyl ester,
j6) procaine,
k6) tetracaine,
l6) chloroprocaine,
m6) oxyprocaine,
n6) mepivacaine,
o6) piperocaine,
p6) pramoxine, and
q6) combinations thereof.

7. The formulation of claim 6
wherein said cocaine (a6) comprises cocaine hydrochloride;
wherein said lignocaine (b6) comprises lignocaine hydrochloride;
wherein said bupivicaine(c6) comprises bupivicaine hydrochloride;
wherein said oxethazaine (d6) comprises oxethazaine hydrochloride,
wherein said dibucaine (e6) comprises dibucaine hydrochloride;
wherein said lidocaine (f6) comprises lidocaine hydrochloride;
wherein said diclonine (h6) comprises dyclonine hydrochloride;
wherein said p-buthylaminobenzoic acid 2-(diethylamino) ethyl ester (i6) comprises p-buthylaminobenzoic acid 2-(diethylamino) ethyl ester hydrochloride;
wherein said procaine (j6) comprises procaine hydrochloride;
wherein said tetracaine (k6) comprises tetiacaine hydrochloride:
wherein said chloroprocaine (l6) comprises chloroprocaine hydrochloride;
wherein said oxyprocaine (m6) comprises oxyprocaine hydrochloride;
wherein said mepivacaine (n6) comprises mepivacaine hydrochloride;
wherein said piperocaine (o6) comprises piperocaine hydrochloride; and
wherein said pramoxine (p6) comprises pramoxine hydrochloride.

8. The formulation of claim 1 wherein said locally acting anesthetic (a1) comprises benzocaine.

9. The formulation of claim 1 wherein said locally acting anesthetic (a1) comprises dyclonine.

10. The formulation of claim 1 wherein said locally acting anesthetic (a1) comprises dyclonine hydrochloride.

11. The formulation of claim 1 wherein said locally acting anesthetic (a1) comprises benzocaine and dyclonine.

12. The formulation of claim 1 wherein said locally acting anesthetic (a1) comprises benzocaine and dyclonine hydrochloride.

13. The formulation of claim 1 wherein said antacid (b1) is an alkaline buffering agent.

14. The formulation of claim 1 wherein said antacid is selected from the group consisting of:
a14) aluminum carbonate,
b14) aluminum hydroxide,
c14) aluminum phosphate,
d14) aluminum citrate,
e14) dihydroxyaluminum sodium carbonate,
f14) aluminum magnesium glycinate,
g14) dihydroxyaluminum aminoacetic acid,
h14) bismuth aluminate,
i14) bismuth carbonate,
j14) bismuth subcarbonate,
k14) bismuth subgallate,
l14) bismuth subnitrate,
m14) calcium carbonate.
n14) calcium hydroxide,
o14) calcium phosphate,
p14) calcium citrate,
q14) activated sulfate,
r14) magnesium aluminate,
s14) magnesium aluminosilicates,
t14) magnesium carbonate,
u14) magnesium glycinate,
v14) magnesium hydroxide,
w14) magnesium oxide.
x14) magnesium trisilicate,
y14) potassium carbonate,
z14) potassium phosphate,
aa14) potassium citrate,
bb14) sodium carbonate,
cc14) sodium bicarbonate,
dd14) sodium phosphate,
ee14) sodium citrate, and
ff14) mixtures thereof.

15. The formulation of claim 14
wherein said aluminum carbonate (a14) comprises aluminum hydroxy carbonate;
wherein said dihydroxyaluminum aminoacetic acid (g14) comprises dihydroxyaluminum aminoacetate;
wherein said calcium citrate (p14) comprises calcium citrate malate; and
wherein said magnesium aluminate (r14) comprises hydrated magnesium aluminate.

16. The formulation of claim 1 wherein said formulation is provided in a dosage form selected from the group consisting of:
a16) an elixir,
b16) a liquid,
c16) a solution,
d16) a suspension.
e16) an emulsion,
f16) a tablet,
g16) a capsule,
h16) a caplet,
i16) a lozenge,
j16) a bead,
k16) a powder,
l16) a granule,
m16) a cachet,
n16) a douche,
o16) a suppository,
p16) a cream,
q16) a topical.
r16) an inhalant,
s16) a patch,
t16) an implant,
u16) an ingestible,
v16) an injectable,
w16) an infusion,
x16) a food,
y16) a sustained release, and
z16) combinations thereof.

17. The formulation of claim 16
wherein said tablet (f16) is selected from the group consisting of:
a17) a compressed tablet,
b17) a film coated tablet,
c17) a chewable tablet,
d17) a quick dissolve tablet,
e17) an effervescent tablet,
f17) a multi-layer tablet, and
g17) a bi-layer tablet;
wherein said capsule (g16) is selected from the group consisting of:
h17) a soft gelatin capsule, and
i17) a hard gelatin capsule;
wherein said lozenge (i16) comprises a chewable lozenge;
wherein said granule (l16) comprises a dispersible granule;
wherein said inhalant (r16) is selected from the group consisting of:
j17) an aerosol inhalant, and
k17) a particle inhalant;
wherein said implant (t16) comprises a depot implant; and
wherein said food (x16) is selected from the group consisting of:
l17) a bar,
m17) a cereal,
n17) a chewing gum,
o17) an animal feed, and
p17) a drink;
wherein said sustained release dosage form is selected from the group consisting of:
q17) a sustained release capsule,
r17) a sustained release granule.
s17) a sustained release tablet.

18. The formulation of claim 1 wherein said locally acting anesthetic (a1) is provided in an amount from about 0.01% to about 50% by weight based on a total weight of said formulation.

19. The formulation of claim 18 wherein said locally acting anesthetic (a1) is provided in an amount from about 0.1% to about 2.5% by weight based on a total weight of said formulation.

20. The formulation of claim 19 wherein said locally acting anesthetic (a1) is provided in an amount from about 0.25% to about 10% by weight based on a total weight of said formulation.

21. The formulation of claim 20 wherein said locally acting anesthetic (a1) is provided in an amount from about 0.5% to about 5% by weight based on a total weight of said formulation.

22. The formulation of claim 21 wherein said locally acting anesthetic (a1) is provided in an amount from about 1% to about 2% by weight based on a total weight of said formulation.

23. The formulation of claim 1 wherein said antacid (b1) is provided in an amount from about 1 mEq to about 50 mEq by weight based on a total weight of said formulation.

24. The formulation of claim 23 wherein said antacid (b1) is provided in an amount from about 5 mEq to about 40 mEq by weight based on a total weight of said formulation.

25. The formulation of claim 24 wherein said antacid (b1) is provided in an amount from about 10 mEq to about 30 mEq by weight based on a total weight of said formulation.

26. The formulation of claim 25 wherein said antacid (b1) is provided in an amount from about 15 mEq to about 25 mEq by weight based on a total weight of said formulation.

27. The formulation of claim 1 further comprising a taste enhancer.

28. The formulation of claim 27 wherein said taste enhancer is selected from the group consisting of: acesulfame-K, aspartame, benzaldehyde, citric acid, corn syrup. fructose, glucose, maltol, mannitol, menthol, monosodium glutamate, saccharin, saccharin sodium, sodium chloride, sorbitol, sucralose, sucrose, vanillin, and combinations thereof.

29. The formulation of claim 1 further comprising a therapeutically effective amount of a drug used to treat a gastrointestinal disorder.

30. The formulation of claim 29 wherein said therapeutically effective drug is selected from the group consisting of:
a30) an H2 blocker;
b30) a proton pump inhibitor;
c30) an antispasm/muscle relaxing agent;
d30) a prokinetic and gastrokinetic agent;
e30) an antifoaming agent;
f30) an anticholinergic agent; and
g30) combinations thereof.

31. The formulation in claim 30
wherein said H2 blocker (a30) is selected from the group consisting of:
a31) famotidine;
b31) cimetidine;
c31) ranitidine;
d31) nizatidine; and
wherein said proton pump inhibitor (b30) is selected from the group consisting of:
e31) omeprazole;
f31) lanoprazole;
g31) pantoprozole,
h31) esomeprazole;
i31) rabeprazole; and
wherein said antispasm/muscle relaxing agent (c30) is selected from the group consisting of:
j31) baclofen; and
k31) 4-amino-3-(4-chloropheyl)-butanoic acid; and
wherein said prokinetic and gastrokinetic agent (d30) comprises:
j31) metaclopramide;
wherein said antifoaming agent (e30) is selected from the group consisting of:
m31) sucrafate; and
n31) carafate; and
wherein said anticholinergic agent (f30) comprises
o32) clidinium.

32. The formulation of claim 1 further comprising a pharmaceutically acceptable bioadhesive.

33. The formulation of claim 32 wherein said pharmaceutically acceptable bioadhesive is selected from the group consisting of: a cellulostic derivative, a polysacchalide, a polypeptide, a synthetic polymer, a vinyl and an acrylic derivative, a polyethylene oxide, a polyethylene glycol; and combinations thereof.

34. The formulation of claim 32 wherein said bioadhesive binds to the lining of a gastrointestinal tract.

35. The formulation of claim 32 wherein said bioadhesive changes viscosity with a change in pH.

36. The formulation of claim 32 wherein said bioadhesive increases viscosity, with an increase in pH.

37. The formulation of claim 32 wherein said bioadhesive increases Viscosity with a decrease in pH.

38. The formulation of claim 32 wherein said bioadhesive adheres to the upper or lower esophageal sphincter.

39. The formulation of claim 1 wherein said formulation provides symptomatic relief of symptoms associated with said gastrointestinal disorder.

40. A formulation for treating a gastrointestinal disorder comprising:
a40) at least two locally acting anesthetics.

41. The formulation of claim 40 wherein said gastrointestinal disorder is selected from the group consisting of:
a41) reflux,
b41) ulcer,
c41) nausea,
d41) gastritis,
e41) dyspepsia,
f41) abrasion to gastrointestinal tract,
g41) heart burn,
h41) hiatal hernia,
i41) gastrointestinal abscess,
j41) inflammatory bowel disease.
k41) colitis,
l41) Crohn's disease,
m41) ileitis,
n41) ileocolitis,
o41) ulcerative proctitis,
p41) irritable bowel syndrome,
q41) gastroenteritis,
r41) diverticulitis,
s41) diverticulosis, and
t41) combinations thereof.

42. The formulation of claim 41
wherein said reflux (a41) is selected from the group consisting of:
a42) gastroesophageal reflux disease (GERD),
b42) reflux esophagitis,
c42) reflux laryngitis,
d42) acid reflux; and
wherein said ulcer (b41) is selected from the group consisting of:
e42) esophageal ulcer,
f42) gastric peptic ulcer, and
g42) duodenal peptic ulcer; and
wherein said abrasion (e41) to gastrointestinal tract is selected from the group consisting of:
h42) scrapes,
i42) puncture, and
j42) surgical; and
wherein said colitis (j41) comprises ulcerative colitis.

43. The formulation of claim 41 wherein said gastrointestinal disorder is gastroesophageal reflux disease (GERD).

44. The formulation of claim 41 wherein said gastrointestinal disorder is acid reflux.

45. The formulation of claim 40 wherein said locally acting anesthetics (a40) are selected from the group consisting of:
a45) cocaine,
b45) lignocaine,
c45) bupivicaine,
d45) oxethazaine,
e45) dibucaine,
f45) lidocaine,
g45) benzocaine,
h45) dyclonine,
i45) p-buthylaminobenzoic acid 2-(diethylamino) ethyl ester.
j45) procaine,
k45) tetracaine,
l45) chloroprocaine,
m45) oxyprocaine,
n45) mepivacaine,
o45) piperocaine,
p45) pramoxine, and
q45) combinations thereof.

46. The formulation of claim 45
wherein said cocaine (a45) comprises cocaine hydrochloride;
wherein said lignocaine (b45) comprises lignocaine hydrochloride;
wherein said bupivicaine (c45) comprises bupivicaine hydrochloride,
wherein said oxethazaine (d45) comprises oxethazaine hydrochloride;
wherein said dibucaine (e45) comprises dibucaine hydrochloride;
wherein said lidocaine (f45) comprises lidocaine hydrochloride;
wherein said dyclonine (h45) comprises dyclonine hydrochloride.
wherein said p-buthylaminobenzoic acid 2-(diethylamino) ethyl ester (i45) comprises p-buthylaminobenzoic acid 2-(diethylamino) ethyl ester hydrochloride;
wherein said procaine (j45) comprises procaine hydrochloride;
wherein said tetracaine (k45) comprises tetracaine hydrochloride:
wherein said chloroprocaine (l45) comprises chloroprocaine hydrochloride:
wherein said oxyprocaine (m45) comprises oxyprocaine hydrochloride;
wherein said mepivacaine (n45) comprises mepivacaine hydrochloride;
wherein said piperocaine (o45) comprises piperocaine hydrochloride; and
wherein said pramoxine (p45) comprises pramoxine hydrochloride.

47. The formulation of claim 40 wherein said locally acting anesthetics (a40) comprise benzocaine and dyclonine hydrochloride.

48. The formulation of claim 40 wherein said locally acting anesthetics (a40) comprise benzocaine and dyclonine.

49. The formulation of claim 40 wherein said formulation is provided in a dosage form selected from the group consisting of, an elixir, a liquid, a solution, a suspension, an emulsion, a tablet, a capsule, a caplet, a lozenge, a bead, a powder, a granule, a cachet, a douche, a suppository, a cream, a topical, an inhalant, a patch, an implant, an ingestible, an injectable, an infusion, a food, a sustained release, and combinations thereof.

50. The formulation of claim 49
wherein said tablet is selected from the group consisting of: a compressed tablet, a film coated tablet, a chewable tablet, a quick dissolve tablet, an effervescent tablet, a multi-layer tablet, a bi-layer tablet;
wherein said capsule is selected from the group consisting of: a soft gelatin capsule, a hard gelatin capsule;
wherein said lozenge comprises a chewable lozenge;
wherein said granule comprises a dispersible granule;
wherein said inhalant is selected from the group consisting of: an aerosol inhalant, a particle inhalant;
wherein said implant comprises a depot implant;
wherein said food is selected from the group consisting of: a bar, a cereal, a chewing gum, a drink, and an animal feed, and
wherein said sustained release dosage form is selected from the group consisting of: a sustained release capsule, a sustained release granule, and a sustained release tablet.

51. The formulation of claim 40 wherein said formulation comprises:
a51) a first locally acting anesthetic, and
b51) a second locally acting anesthetic.

52. The formulation of claim 51 wherein said first locally acting anesthetic (a51) is provided in an amount from about 0.01% to about 50% by weight based on a total weight of said formulation.

53. The formulation of claim 52 wherein said first locally acting anesthetic (a51) is provided in an amount from about 0.1% to about 25% by weight based on a total weight of said formulation.

54. The formulation of claim 53 wherein said first locally acting anesthetic (a51) is provided in an amount from about 0.25% to about 10% by weight based on a total weight of said formulation.

55. The formulation of claim 54 wherein said first locally acting anesthetic (a51) is provided in an amount from about 0.5% to about 5% by weight based on a total weight of said formulation.

56. The formulation of claim 55 wherein said first locally acting anesthetic (a51) is provided in an amount from about 1% to about 2% by weight based on a total weight of said formulation.

57. The formulation of claim 51 wherein said second locally acting anesthetic (b51) is provided in an amount from about 0.01% to about 50% by weight based on a total weight of said formulation.

58. The formulation of claim 57 wherein said second locally acting anesthetic (b51) is provided in an amount from about 0.1% to about 2.5% by weight based on a total weight of said formulation.

59. The formulation of claim 58 wherein said second locally acting anesthetic (b51) is provided in an amount from about 0.25% to about 10% by weight based on a total weight of said formulation.

60. The formulation of claim 59 wherein said second locally acting anesthetic (b51) is provided in an amount from about 0.5% to about 5% by weight based on a total weight of said formulation.

61. The formulation of claim 59 wherein said second locally acting anesthetic (b51) is provided in an amount from about 1% to about 2% by weight based on a total weight of said formulation.

62. The formulation of claim 40 further comprising a taste enhancer.

63. The formulation of claim 62 wherein said taste enhancer is selected from the group consisting of: acesulfame-K, aspartame, benzaldehyde, citric acid, corn syrup, fructose, glucose, maltol, mannitol, menthol, monosodium glutamate, saccharin, saccharin sodium, sodium chloride, sorbitol, sucralose, sucrose, vanillin, and combinations thereof.

64. The formulation of claim 40 further comprising a therapeutically effective amount of a drug used to treat a gastrointestinal disorder.

65. The formulation of claim 64 wherein said therapeutically effective drug is selected from the group consisting of:
a65) an H2 blocker;
b65) a proton pump inhibitor;
c65) an antispasm/muscle relaxing agent;
d65) a prokinetic and gastrokinetic agent;
e65) an antifoaming agent;
f65) an anticholinergic agent; and
g65) combinations thereof.

66. The formulation in claim 65
wherein said H2 blocker (a65) is selected from the group consisting of:
a66) famotidine;
b66) cimetidine;
c66) ranitidine;
d66) nizatidine; and
wherein said proton pump inhibitor (b65) is selected from the group consisting of:
e66) omeprazole;
f66) lanoprazole;
g66) pantoprozole;
h66) esomeprazole;
i66) rabeprazole; and
wherein said antispasm/muscle relaxing agent (c65) is selected from the group consisting of:
j66) baclofen; and
k66) 4-amino-3-(4-chloropheyl)-butanoic acid; and
wherein said prokinetic and gastrokinetic agent (d65) is selected from the group consisting of:
l66) metaclopramide;
wherein said antifoaming agent (e65) is selected from the group consisting of:
m66) sucrafate; and
n66) carafate; and
wherein said anticholinergic agent (f65) comprises:
o66) clidinium.

67. The formulation of claim 40 further comprising a pharmaceutically acceptable bioadhesive.

68. The formulation of claim 67 wherein said pharmaceutically acceptable bioadhesive is selected from the group consisting of: a cellulostic derivative, a polysaccharide. a polypeptide, a synthetic polymer, a vinyl and an acrylic derivative, a polyethylene oxide, a polyethylene glycol, and combinations thereof.

69. The formulation of claim 67 wherein said bioadhesive binds to the lining of a gastrointestinal tract.

70. The formulation of claim 67 wherein said bioadhesive changes viscosity with a change in pH.

71. The formulation of claim 67 wherein said bioadhesive increases viscosity with an increase in pH.

72. The formulation of claim 67 wherein said bioadhesive increases viscosity with a decrease in pH.

73. The formulation of claim 67 wherein said bioadhesive adheres to the upper or lower esophageal sphincter.

74. The formulation of claim 40 further comprising an antacid.

75. The formulation of claim 40 wherein said formulation provides symptomatic relief of symptoms associated with said gastroesophageal disease.

76. A method for treating a gastrointestinal disorder in a patient in need thereof said method comprising the step of:
a76) administering to said patient a therapeutically effective amount of a formulation comprising a locally acting anesthetic.

77. A method for treating a gastrointestinal disorder in a patient in need thereof, said method comprising the step of:
a77) administering to said patient a therapeutically effective amount of said formulation of claim 1.

78. A method for treating a gastrointestinal disorder in a patient in need thereof, said method comprising the step of:
a78) administering to said patient a therapeutically effective amount of said formulation of claim 29.

79. A method for treating a gastrointestinal disorder in a patient in need thereof, said method comprising the step of:
a79) administering to said patient a therapeutically effective amount of said formulation of claim 40.

80. A method for treating a gastrointestinal disorder in a patient in need thereof, said method comprising the step of:
a80) administering to said patient a therapeutically effective amount of said formulation of claim 64.

81. The method of claim 77 wherein said administering step comprises a route of administration selected from the group consisting of:
a81) oral,
b81) rectal,
c81) surgical, or
d81) combinations thereof.

82. The method of claim 81 wherein said surgical (c81) route of administration comprises a surgical implant.

83. The method of claim 82 wherein said surgical implant comprises a slow release dosage implant.

84. The method of claim 77 wherein said gastrointestinal disorder is selected from the group consisting of:
a84) reflux,
b84) ulcer,
c84) gastritis,
d84) nausea,
e84) dyspepsia,
f84) abrasion to gastrointestinal tract;
g84) heart burn,
h84) hiatal hernia,
i84) gastrointestinal abscess,
j84) inflammatory bowel disease,
k84) colitis,
l84) Crohn's disease,
m84) ileitis,
n84) ileocolitis,
o84) ulcerative proctitis,
p84) irritable bowel syndrome,
q84) gastroenteritis,
r84) diverticulitis,
s84) diverticulosis, and
t84) combinations thereof.

85. The method of claim 84
wherein said reflux (a84) is selected from the group consisting of:
a85) gastroesophageal reflux disease (GERD),
b85) reflux esophagitis,
c85) reflux laryngitis,
d85) acid reflux; and
wherein said ulcer (b84) is selected from the group consisting of:
e85) esophageal ulcer,
f85) gastric peptic ulcer, and
g85) duodenal peptic ulcer; and
wherein said abrasion (e84) to gastrointestinal tract is selected from the group consisting of:
h85) scrapes,
i85) puncture, and
j85) surgical; and
wherein said colitis (j84) comprises ulcerative colitis.

86. The method of claim 85 wherein said gastrointestinal disease is gastrointestinal reflux disease (GERD).

87. The method of claim 85 wherein said gastrointestinal disease is acid reflux.

88. The method of claim 79 wherein said gastrointestinal disorder is selected from the group consisting of:
a88) reflux,
b88) ulcer,
c88) gastritis,
d88) nausea,
e88) dyspepsia,
f88) abrasion to gastrointestinal tract;
g88) heart burn,
h88) hiatal hernia,
i88) gastrointestinal abscess,
j88) inflammatory bowel disease,
k88) colitis,
l88) Crohn's disease,
m88) ileitis,
n88) ileocolitis,
o88) ulcerative proctitis,
p88) irritable bowel syndrome,
q88) gastroenteritis,
r88) diverticulitis,
s88) diverticulosis, and
t88) combinations thereof.

89. The method of claim 88
wherein said reflux (a88) is selected from the group consisting of:
a89) gastroesophageal reflux disease (GERD),
b89) reflux esophagitis.
c89) reflux laryngitis,
d89) acid reflux; and
wherein said ulcer (b88) is selected from the group consisting of:
e89) esophageal ulcer,
f89) gastric peptic ulcer, and
g89) duodenal peptic ulcer; and
wherein said abrasion (e88) to gastrointestinal tract is selected from the group consisting of:
h89) scrapes,
i89) puncture, and
j89) surgical; and
wherein said colitis (j88) comprises ulcerative colitis.

90. The method of claim 89 wherein said gastrointestinal disease is gastrointestinal reflux disease (GERD).

91. The method of claim 89 wherein said gastrointestinal disease is acid reflux.

92. A formulation for treating a gastrointestinal disorder consisting essentially of:
a92) a locally acting anesthetic, and
b92) an antacid.

93. A formulation for treating a gastrointestinal disorder consisting of:
a93) a locally acting anesthetic, and
b93) an antacid.

94. A formulation for treating a gastrointestinal disorder consisting of:
a94) at least two locally acting anesthetics.

95. A formulation for treating a gastrointestinal disorder consisting essentially of:
a95) at least two locally acting anesthetics.
